SEQUENCE LISTING (391 sequences)

SEQ ID NO: 1 - DNA, 366 bp, synthetic construct
  misc_feature 1..366: note = Synthetic
  source 1..366: mol_type = other DNA
caggtgcagctggtgcagtctgggggaggcttggtccagcctggggggtccctgagactctcctgtgcagcctctggattcacctttaacacctatgccataagctgggtccgccaggctccagggaaggggctggaatgggtctcaactattactggtagtggttataacacatactacgcagactccgtgaagggccggttcaccatctccagagacaattccaagaacacactgtatctacagatgagcagcctgagagccgaggacacggccgtattttactgtgcgaaagactctcggtataactggaattacggaatttttgactactggggccagggaaccacggtcaccgtctcctca

SEQ ID NO: 2 - protein, 122 aa, synthetic construct
  REGION 1..122: note = Synthetic
QVQLVQSGGGLVQPGGSLRLSCAASGFTFNTYAISWVRQAPGKGLEWVSTITGSGYNTYYADSVKGRFTISRDNSKNTLYLQMSSLRAEDTAVFYCAKDSRYNWNYGIFDYWGQGTTVTVSS

SEQ ID NO: 3 - DNA, 24 bp, synthetic construct
  misc_feature 1..24: note = Synthetic
  source 1..24: mol_type = other DNA
ggattcacctttaacacctatgcc

SEQ ID NO: 4 - protein, 8 aa, synthetic construct
  REGION 1..8: note = Synthetic
GFTFNTYA

SEQ ID NO: 5 - DNA, 24 bp, synthetic construct
  misc_feature 1..24: note = Synthetic
  source 1..24: mol_type = other DNA
attactggtagtggttataacaca

SEQ ID NO: 6 - protein, 8 aa, synthetic construct
  REGION 1..8: note = Synthetic
ITGSGYNT

SEQ ID NO: 7 - DNA, 45 bp, synthetic construct
  misc_feature 1..45: note = Synthetic
  source 1..45: mol_type = other DNA
gcgaaagactctcggtataactggaattacggaatttttgactac

SEQ ID NO: 8 - protein, 15 aa, synthetic construct
  REGION 1..15: note = Synthetic
AKDSRYNWNYGIFDY

SEQ ID NO: 9 - DNA, 324 bp, synthetic construct
  misc_feature 1..324: note = Synthetic
  source 1..324: mol_type = other DNA
gacatccagatgacccagtctccagccaccctgtctgtgtctccaggggaaagagccaccctctcctgcagggccagtcagagtgttagcagcaacttagcctggtaccatcagaaacctggccaggctcccaggctcctcatctatggtgtatccaccagggccactggtatcccagccaggttcagtggcaatgggtctgggacagagttcactctcaccatcagcagcctgcagtctgaagattttgcagtttattactgtcagcagcataataactggccgctcactttcggcggagggaccaaggtggagatcaaacga

SEQ ID NO: 10 - protein, 108 aa, synthetic construct
  REGION 1..108: note = Synthetic
DIQMTQSPATLSVSPGERATLSCRASQSVSSNLAWYHQKPGQAPRLLIYGVSTRATGIPARFSGNGSGTEFTLTISSLQSEDFAVYYCQQHNNWPLTFGGGTKVEIKR

SEQ ID NO: 11 - DNA, 18 bp, synthetic construct
  misc_feature 1..18: note = Synthetic
  source 1..18: mol_type = other DNA
cagagtgttagcagcaac

SEQ ID NO: 12 - protein, 6 aa, synthetic construct
  REGION 1..6: note = Synthetic
QSVSSN

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
cagcagcataataactggccgctcact

SEQ ID NO: 16 - protein, 9 aa, synthetic construct
  REGION 1..9: note = Synthetic
QQHNNWPLT

SEQ ID NO: 17 - DNA, 375 bp, synthetic construct
  misc_feature 1..375: note = Synthetic
  source 1..375: mol_type = other DNA
caggtgcagctggtgcagtcgggcccaggactggtgaagccttcggagaccctgtccctcacctgcactgtctctggtggctccatcagcaatagtaattactactggggctggatccgccagcccccagggaaggggctggagtggattgggactacctattatagtgggaccacctactacaacccgtccctcaagagtcgagtcaccatatccgtagacacgtccaagaaccagttctccctgaagctgagctctgtgaccgccgcagacacggctgtgtattactgtgcgagagattattatgatagtagtggttattattacaactggttcgatccctggggccagggaaccacggtcaccgtctcctca

SEQ ID NO: 18 - protein, 125 aa, synthetic construct
  REGION 1..125: note = Synthetic
QVQLVQSGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 19 - DNA, 30 bp, synthetic construct
  misc_feature 1..30: note = Synthetic
  source 1..30: mol_type = other DNA
ggtggctccatcagcaatagtaattactac

SEQ ID NO: 20 - protein, 10 aa, synthetic construct
  REGION 1..10: note = Synthetic
GGSISNSNYY

SEQ ID NO: 21 - DNA, 21 bp, synthetic construct
  misc_feature 1..21: note = Synthetic
  source 1..21: mol_type = other DNA
acctattatagtgggaccacc

SEQ ID NO: 22 - protein, 7 aa, synthetic construct
  REGION 1..7: note = Synthetic
TYYSGTT

SEQ ID NO: 23 - DNA, 51 bp, synthetic construct
  misc_feature 1..51: note = Synthetic
  source 1..51: mol_type = other DNA
gcgagagattattatgatagtagtggttattattacaactggttcgatccc

SEQ ID NO: 24 - protein, 17 aa, synthetic construct
  REGION 1..17: note = Synthetic
ARDYYDSSGYYYNWFDP

SEQ ID NO: 25 - DNA, 324 bp, synthetic construct
  misc_feature 1..324: note = Synthetic
  source 1..324: mol_type = other DNA
gacatccagttgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacagaattcagtctcacactcagcagcctgcagcctgaagattttgcaacttatttctgtctacagcatcatatttacccgtggacgttcggccaagggaccaagctggagatcaaacga

SEQ ID NO: 26 - protein, 108 aa, synthetic construct
  REGION 1..108: note = Synthetic
DIQLTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKLEIKR

SEQ ID NO: 27 - DNA, 18 bp, synthetic construct
  misc_feature 1..18: note = Synthetic
  source 1..18: mol_type = other DNA
caggacattagaaatgat

SEQ ID NO: 28 - protein, 6 aa, synthetic construct
  REGION 1..6: note = Synthetic
QDIRND

SEQ ID NO: 29-30 - skipped (2 entries)

SEQ ID NO: 31 - DNA, 27 bp, synthetic construct
  misc_feature 1..27: note = Synthetic
  source 1..27: mol_type = other DNA
ctacagcatcatatttacccgtggacg

SEQ ID NO: 32 - protein, 9 aa, synthetic construct
  REGION 1..9: note = Synthetic
LQHHIYPWT

SEQ ID NO: 33 - DNA, 375 bp, synthetic construct
  misc_feature 1..375: note = Synthetic
  source 1..375: mol_type = other DNA
caagttcagctggtggagtcgggcccaggactggtgaagccttcggagaccctgtccctcacctgcactgtctatggtggctccatcagcagtggtaattactactggggctggatccgccagcccccagggaagggactggagtggattgggactatctattatagtggaagcgcctactacaacccgtccctcaagagtcgagtcaccatgtccgtagacacgtccaagaaccagttctccctgaaactgagctctgtgaccgccgcagacacggctgtttattactgtgtgagagattactatgatagtagtggtcattattacaactggttcgacccctggggccagggaaccacggtcaccgtctcctca

SEQ ID NO: 34 - protein, 125 aa, synthetic construct
  REGION 1..125: note = Synthetic
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 35 - DNA, 30 bp, synthetic construct
  misc_feature 1..30: note = Synthetic
  source 1..30: mol_type = other DNA
ggtggctccatcagcagtggtaattactac

SEQ ID NO: 36 - protein, 10 aa, synthetic construct
  REGION 1..10: note = Synthetic
GGSISSGNYY

SEQ ID NO: 37 - DNA, 21 bp, synthetic construct
  misc_feature 1..21: note = Synthetic
  source 1..21: mol_type = other DNA
atctattatagtggaagcgcc

SEQ ID NO: 38 - protein, 7 aa, synthetic construct
  REGION 1..7: note = Synthetic
IYYSGSA

SEQ ID NO: 39 - DNA, 51 bp, synthetic construct
  misc_feature 1..51: note = Synthetic
  source 1..51: mol_type = other DNA
gtgagagattactatgatagtagtggtcattattacaactggttcgacccc

SEQ ID NO: 40 - protein, 17 aa, synthetic construct
  REGION 1..17: note = Synthetic
VRDYYDSSGHYYNWFDP

SEQ ID NO: 41 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcaggacattagacatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttacccgtggacgttcggccaagggaccaaggtggagatcaaacga

SEQ ID NO: 42 - protein, 108 aa, synthetic construct
  REGION 1..108: note = Synthetic
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 43 - DNA, 18 bp, synthetic construct
  misc_feature 1..18: note = Synthetic
  source 1..18: mol_type = other DNA
caggacattagacatgat

SEQ ID NO: 44 - protein, 6 aa, synthetic construct
  REGION 1..6: note = Synthetic
QDIRHD

SEQ ID NO: 45-46 - skipped (2 entries)

SEQ ID NO: 47 - DNA, 27 bp, synthetic construct
  misc_feature 1..27: note = Synthetic
  source 1..27: mol_type = other DNA
ctacagcataatacttacccgtggacg

SEQ ID NO: 48 - protein, 9 aa, synthetic construct
  REGION 1..9: note = Synthetic
LQHNTYPWT

SEQ ID NO: 49 - DNA, 357 bp, synthetic construct
  misc_feature 1..357: note = Synthetic
  source 1..357: mol_type = other DNA
caggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcgtggatacacctttggggttgactactggggccagggaaccacggtcaccgtctcctca

SEQ ID NO: 50 - protein, 119 aa, synthetic construct
  REGION 1..119: note = Synthetic
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYTFGVDYWGQGTTVTVSS

SEQ ID NO: 51 - DNA, 24 bp, synthetic construct
  misc_feature 1..24: note = Synthetic
  source 1..24: mol_type = other DNA
ggattcaccttcagtagctatagc

SEQ ID NO: 52 - protein, 8 aa, synthetic construct
  REGION 1..8: note = Synthetic
GFTFSSYS

SEQ ID NO: 53 - DNA, 24 bp, synthetic construct
  misc_feature 1..24: note = Synthetic
  source 1..24: mol_type = other DNA
attagtagtagtagtagttacata

SEQ ID NO: 54 - protein, 8 aa, synthetic construct
  REGION 1..8: note = Synthetic
ISSSSSYI

SEQ ID NO: 55 - DNA, 36 bp, synthetic construct
  misc_feature 1..36: note = Synthetic
  source 1..36: mol_type = other DNA
gcgagagatcgtggatacacctttggggttgactac

SEQ ID NO: 56 - protein, 12 aa, synthetic construct
  REGION 1..12: note = Synthetic
ARDRGYTFGVDY

SEQ ID NO: 57 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaaggtggagatcaaacga

SEQ ID NO: 58 - protein, 108 aa, synthetic construct
  REGION 1..108: note = Synthetic
AIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKVEIKR

SEQ ID NO: 59 - DNA, 18 bp, synthetic construct
  misc_feature 1..18: note = Synthetic
  source 1..18: mol_type = other DNA
cagggcattagaaatgat

SEQ ID NO: 60 - protein, 6 aa, synthetic construct
  REGION 1..6: note = Synthetic
QGIRND

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
ctacagcataatagttacccgtacact

SEQ ID NO: 64 - protein, 9 aa, synthetic construct
  REGION 1..9: note = Synthetic
LQHNSYPYT

SEQ ID NO: 65 - DNA, 375 bp, synthetic construct
  misc_feature 1..375: note = Synthetic
  source 1..375: mol_type = other DNA
caggtgcagctggtgcagtcgggcccaggactggtgaagccttcggagaccctgtccctcacctgcactgtctctggtggctccatcatcacttatagttactactggggctggatccgccagcccccagggaaggggctggagtggattgggactatccatcatagtgggagcacctactacaacccgtccctcaagagtcgagtcaccatatccgtagacacgtccaagaaccagttctccctgacactgagttctgtgaccgccgcagacacggctgtgtattactgtgcgagagactactatgatagtagtggttattattataactggttcgacccctggggccagggaaccatggtcaccgtctcctca

SEQ ID NO: 66 - protein, 125 aa, synthetic construct
  REGION 1..125: note = Synthetic
QVQLVQSGPGLVKPSETLSLTCTVSGGSIITYSYYWGWIRQPPGKGLEWIGTIHHSGSTYYNPSLKSRVTISVDTSKNQFSLTLSSVTAADTAVYYCARDYYDSSGYYYNWFDPWGQGTMVTVSS

SEQ ID NO: 67 - DNA, 30 bp, synthetic construct
  misc_feature 1..30: note = Synthetic
  source 1..30: mol_type = other DNA
ggtggctccatcatcacttatagttactac

SEQ ID NO: 68 - protein, 10 aa, synthetic construct
  REGION 1..10: note = Synthetic
GGSIITYSYY

SEQ ID NO: 69 - DNA, 21 bp, synthetic construct
  misc_feature 1..21: note = Synthetic
  source 1..21: mol_type = other DNA
atccatcatagtgggagcacc

SEQ ID NO: 70 - protein, 7 aa, synthetic construct
  REGION 1..7: note = Synthetic
IHHSGST

SEQ ID NO: 71 - DNA, 51 bp, synthetic construct
  misc_feature 1..51: note = Synthetic
  source 1..51: mol_type = other DNA
gcgagagactactatgatagtagtggttattattataactggttcgacccc

SEQ ID NO: 72 - protein, 17 aa, synthetic construct
  REGION 1..17: note = Synthetic
ARDYYDSSGYYYNWFDP

SEQ ID NO: 73 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttccccgtggacgttcggccaagggaccaaggtggagatcaaacga

SEQ ID NO: 74 - protein, 108 aa, synthetic construct
  REGION 1..108: note = Synthetic
AIQMTQSPSSLSASVGDRVTITCRASQGIRNDLGWYQQKPGKAPKRLIYAASSLQSGVPSRFSGSGSGTEFTLTISSLQPEDFATYYCLQHNSSPWTFGQGTKVEIKR

SEQ ID NO: 75 - DNA, 18 bp, synthetic construct
  misc_feature 1..18: note = Synthetic
  source 1..18: mol_type = other DNA
cagggcattagaaatgat

SEQ ID NO: 76 - protein, 6 aa, synthetic construct
  REGION 1..6: note = Synthetic
QGIRND

SEQ ID NO: 77-78 - skipped (2 entries)

SEQ ID NO: 79 - DNA, 27 bp, synthetic construct
  misc_feature 1..27: note = Synthetic
  source 1..27: mol_type = other DNA
ctacagcataatagttccccgtggacg

SEQ ID NO: 80 - protein, 9 aa, synthetic construct
  REGION 1..9: note = Synthetic
LQHNSSPWT

SEQ ID NO: 81 - DNA, 369 bp, synthetic construct
  misc_feature 1..369: note = Synthetic
  source 1..369: mol_type = other DNA
caggtgcacttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtacacacacctcccgttataactggcactacggcttccttgactactggggccagggaaccacggtcaccgtctcctca

SEQ ID NO: 82 - protein, 123 aa, synthetic construct
  REGION 1..123: note = Synthetic
QVHLKESGPTLVKPTQTLTLTCTFSGFSLSTSGVGVGWIRQPPGKALEWLALIYWNDDKRYSPSLKSRLTITKDTSKNQVVLTMTNMDPVDTATYYCTHTSRYNWHYGFLDYWGQGTTVTVSS

SEQ ID NO: 83 - DNA, 30 bp, synthetic construct
  misc_feature 1..30: note = Synthetic
  source 1..30: mol_type = other DNA
gggttctcactcagcactagtggagtgggt

SEQ ID NO: 84 - protein, 10 aa, synthetic construct
  REGION 1..10: note = Synthetic
GFSLSTSGVG

SEQ ID NO: 85 - DNA, 21 bp, synthetic construct
  misc_feature 1..21: note = Synthetic
  source 1..21: mol_type = other DNA
atttattggaatgatgataag

SEQ ID NO: 86 - protein, 7 aa, synthetic construct
  REGION 1..7: note = Synthetic
IYWNDDK

SEQ ID NO: 87 - DNA, 45 bp, synthetic construct
  misc_feature 1..45: note = Synthetic
  source 1..45: mol_type = other DNA
acacacacctcccgttataactggcactacggcttccttgactac

SEQ ID NO: 88 - protein, 15 aa, synthetic construct
  REGION 1..15: note = Synthetic
THTSRYNWHYGFLDY

SEQ ID NO: 89 - DNA, 324 bp, synthetic construct
  misc_feature 1..324: note = Synthetic
  source 1..324: mol_type = other DNA
gccatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacaatataatagttacccgctcactttcggcggagggaccaaggtggaaatcaaacga

SEQ ID NO: 90 - protein, 108 aa, synthetic construct
  REGION 1..108: note = Synthetic
AIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIKR

SEQ ID NO: 91 - DNA, 18 bp, synthetic construct
  misc_feature 1..18: note = Synthetic
  source 1..18: mol_type = other DNA
cagggcattagcaattat

SEQ ID NO: 92 - protein, 6 aa, synthetic construct
  REGION 1..6: note = Synthetic
QGISNY

SEQ ID NO: 93-94 - skipped (2 entries)

SEQ ID NO: 95 - DNA, 27 bp, synthetic construct
  misc_feature 1..27: note = Synthetic
  source 1..27: mol_type = other DNA
caacaatataatagttacccgctcact

SEQ ID NO: 96 - protein, 9 aa, synthetic construct
  REGION 1..9: note = Synthetic
QQYNSYPLT

SEQ ID NO: 97 - DNA, 375 bp, synthetic construct
  misc_feature 1..375: note = Synthetic
  source 1..375: mol_type = other DNA
gaggtgcagctggtgcagttgggcccaggactggtgaagccttcggagaccctgtccctcacctgcactgtctctggtggctccatcagcaatagtaattactactggggctggatccgccagcccccagggaaggggctggagtggattgggactacctattatagtgggaccacctactacaacccgtccctcaagagtcgagtcaccatatccgtagacacgtccaagaaccagttctccctgaagctgagctctgtgaccgccgcagacacggctgtgtattactgtgcgagagattattatgatagtagtggttattattacaactggttcgatccctggggccagggaaccacggtcaccgtctcctca

SEQ ID NO: 98 - protein, 125 aa, synthetic construct
  REGION 1..125: note = Synthetic
EVQLVQLGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 99 - DNA, 30 bp, synthetic construct; identical to SEQ ID NO: 19
  misc_feature 1..30: note = Synthetic
  source 1..30: mol_type = other DNA

SEQ ID NO: 100 - protein, 10 aa, synthetic construct
  REGION 1..10: note = Synthetic
GGSISNSNYY

SEQ ID NO: 101 - DNA, 21 bp, synthetic construct
  misc_feature 1..21: note = Synthetic
  source 1..21: mol_type = other DNA
acctattatagtgggaccacc

SEQ ID NO: 102 - protein, 7 aa, synthetic construct
  REGION 1..7: note = Synthetic
TYYSGTT

SEQ ID NO: 103 - DNA, 51 bp, synthetic construct; identical to SEQ ID NO: 23
  misc_feature 1..51: note = Synthetic
  source 1..51: mol_type = other DNA

SEQ ID NO: 104 - protein, 17 aa, synthetic construct
  REGION 1..17: note = Synthetic
ARDYYDSSGYYYNWFDP

SEQ ID NO: 105 - DNA, 324 bp, synthetic construct
  misc_feature 1..324: note = Synthetic
  source 1..324: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacagaattcagtctcacactcagcagcctgcagcctgaagattttgcaacttatttctgtctacagcatcatatttacccgtggacgttcggccaagggaccaaggtggagatcaaacga

SEQ ID NO: 106 - protein, 108 aa, synthetic construct
  REGION 1..108: note = Synthetic
AIQMTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKVEIKR

SEQ ID NO: 107 - DNA, 18 bp, synthetic construct
  misc_feature 1..18: note = Synthetic
  source 1..18: mol_type = other DNA
caggacattagaaatgat

SEQ ID NO: 108 - protein, 6 aa, synthetic construct
  REGION 1..6: note = Synthetic
QDIRND

SEQ ID NO: 109-110 - skipped (2 entries)

SEQ ID NO: 111 - DNA, 27 bp, synthetic construct
  misc_feature 1..27: note = Synthetic
  source 1..27: mol_type = other DNA
ctacagcatcatatttacccgtggacg

SEQ ID NO: 112 - protein, 9 aa, synthetic construct
  REGION 1..9: note = Synthetic
LQHHIYPWT

SEQ ID NO: 113 - DNA, 355 bp, synthetic construct
  misc_feature 1..355: note = Synthetic
  source 1..355: mol_type = other DNA
caggtgcagctggtggagtctgggggaggcgtggtccagcctgggaggtccctgagactctcctgtgcagcctctggattcaccttcagtcgctatggcattcactgggtccgccaggctccaggcaaggggctggagtgggtggctgttatatcttatgatggaagtgatgaatactatgtagactccgtgaagggccgattcagcatctcccgagacaattccaagaacacgctttatctacaaatgaacagtctgaggcctgcggactcggctgtttattactgtgtgaaaggagatctggaacttggttttgactactggggccagggaaccctggtcaccgtctcctcaa

SEQ ID NO: 114 - protein, 118 aa, synthetic construct
  REGION 1..118: note = Synthetic
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 115 - DNA, 24 bp, synthetic construct
  misc_feature 1..24: note = Synthetic
  source 1..24: mol_type = other DNA
ggattcaccttcagtcgctatggc

SEQ ID NO: 116 - protein, 8 aa, synthetic construct
  REGION 1..8: note = Synthetic
GFTFSRYG

SEQ ID NO: 117 - DNA, 24 bp, synthetic construct
  misc_feature 1..24: note = Synthetic
  source 1..24: mol_type = other DNA
atatcttatgatggaagtgatgaa

SEQ ID NO: 118 - protein, 8 aa, synthetic construct
  REGION 1..8: note = Synthetic
ISYDGSDE

SEQ ID NO: 119 - DNA, 33 bp, synthetic construct
  misc_feature 1..33: note = Synthetic
  source 1..33: mol_type = other DNA
gtgaaaggagatctggaacttggttttgactac

SEQ ID NO: 120 - protein, 11 aa, synthetic construct
  REGION 1..11: note = Synthetic
VKGDLELGFDY

SEQ ID NO: 121 - DNA, 337 bp, synthetic construct
  misc_feature 1..337: note = Synthetic
  source 1..337: mol_type = other DNA
gatattgtgatgactcaggctgcaccctctatacctgtcattccaggagagtcagtatccatgtcctgcaggtctagtaagagtctcctgtacagtaatggacatacttacgtgtattggtttgtgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaaaatctagaatttccgctcacgttcggtgctgggaccaagctggagctgaaac

SEQ ID NO: 122 - protein, 112 aa, synthetic construct
  REGION 1..112: note = Synthetic
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 123 - DNA, 33 bp, synthetic construct
  misc_feature 1..33: note = Synthetic
  source 1..33: mol_type = other DNA
aagagtctcctgtacagtaatggacatacttac

SEQ ID NO: 124 - protein, 11 aa, synthetic construct
  REGION 1..11: note = Synthetic
KSLLYSNGHTY

SEQ ID NO: 125-126 - skipped (2 entries)

SEQ ID NO: 127 - DNA, 27 bp, synthetic construct
  misc_feature 1..27: note = Synthetic
  source 1..27: mol_type = other DNA
atgcaaaatctagaatttccgctcacg

SEQ ID NO: 128 - protein, 9 aa, synthetic construct
  REGION 1..9: note = Synthetic
MQNLEFPLT

SEQ ID NO: 129 - DNA, 355 bp, synthetic construct
  misc_feature 1..355: note = Synthetic
  source 1..355: mol_type = other DNA
caggtgcagctggtggaggcggggggaggcgtggtccagcctgggaggtccctgagactctcctgtgcagcctctggattcaccttcagtcgctatggcattcactgggtccgccaggctccaggcaaggggctggagtgggtggctgttatatcttatgatggaactgatgaatactatgcagactccgtgaagggccgattcaccatctcccgagacaattccaagaacacgctttatctacaaatgaacagtctgagacctgcggactcggctgtatattactgtgcgaaaggagatctggaacttggttttgactactggggccagggaaccctggtcaccgtctcctcaa

SEQ ID NO: 130 - protein, 118 aa, synthetic construct
  REGION 1..118: note = Synthetic
QVQLVEAGGGVVQPGRSLRLSCAASGFTFSRYGIHWVRQAPGKGLEWVAVISYDGTDEYYADSVKGRFTISRDNSKNTLYLQMNSLRPADSAVYYCAKGDLELGFDYWGQGTLVTVSS

SEQ ID NO: 131 - DNA, 24 bp, synthetic construct
  misc_feature 1..24: note = Synthetic
  source 1..24: mol_type = other DNA
ggattcaccttcagtcgctatggc

SEQ ID NO: 132 - protein, 8 aa, synthetic construct
  REGION 1..8: note = Synthetic
GFTFSRYG

SEQ ID NO: 133 - DNA, 24 bp, synthetic construct
  misc_feature 1..24: note = Synthetic
  source 1..24: mol_type = other DNA
atatcttatgatggaactgatgaa

SEQ ID NO: 134 - protein, 8 aa, synthetic construct
  REGION 1..8: note = Synthetic
ISYDGTDE

SEQ ID NO: 135 - DNA, 33 bp, synthetic construct
  misc_feature 1..33: note = Synthetic
  source 1..33: mol_type = other DNA
gcgaaaggagatctggaacttggttttgactac

SEQ ID NO: 136 - protein, 11 aa, synthetic construct
  REGION 1..11: note = Synthetic
AKGDLELGFDY

SEQ ID NO: 137 - DNA, 322 bp, synthetic construct
  misc_feature 1..322: note = Synthetic
  source 1..322: mol_type = other DNA
gccatccggttgacccagtctccatcttccgtgtctgcatctgtgggagacagagtcaccatcacttgtcgggcgagtcaggatattagtatttggttagcctggtatcagcagagtccagggaaagcccctaaactcctgatcaatgttgcatcccgtttgcaaagtggggtcccatcaaggttcagcggcagtggatctgggacagatttcactctcaccatcaacggtctgcagcctgaagattttgtaacttactattgtcaacaggctaacagtttcccgatcaccttcggccaagggacacgactggcgaccaaac

SEQ ID NO: 138 - protein, 107 aa, synthetic construct
  REGION 1..107: note = Synthetic
AIRLTQSPSSVSASVGDRVTITCRASQDISIWLAWYQQSPGKAPKLLINVASRLQSGVPSRFSGSGSGTDFTLTINGLQPEDFVTYYCQQANSFPITFGQGTRLATK

SEQ ID NO: 139 - DNA, 18 bp, synthetic construct
  misc_feature 1..18: note = Synthetic
  source 1..18: mol_type = other DNA
caggatattagtatttgg

SEQ ID NO: 140 - protein, 6 aa, synthetic construct
  REGION 1..6: note = Synthetic
QDISIW

SEQ ID NO: 141-142 - skipped (2 entries)

SEQ ID NO: 143 - DNA, 27 bp, synthetic construct
  misc_feature 1..27: note = Synthetic
  source 1..27: mol_type = other DNA
caacaggctaacagtttcccgatcacc

SEQ ID NO: 144 - protein, 9 aa, synthetic construct
  REGION 1..9: note = Synthetic
QQANSFPIT

SEQ ID NO: 145 - DNA, 358 bp, synthetic construct
  misc_feature 1..358: note = Synthetic
  source 1..358: mol_type = other DNA
caggtgcagctggtgcagtctgggggaggcctggtcaagcctggggggtccctgagactctcctgtgcagcctctggattcatcttcaatacctataccatgaattgggtccgccaggctccagggaaggggctggagtgggtctcatccatcactagtcgtggtacttatatattctactcagactcacttaagggccgattcaccatttccagagacaacgccaataactcactgtttctgcaaatgaacagcctgagagtcgaagacacggctgtttattactgttcgagagatcgtggatacacctttggtcctgactactggggccagggaaccctggtcaccgtctcttcag

SEQ ID NO: 146 - protein, 119 aa, synthetic construct
  REGION 1..119: note = Synthetic
QVQLVQSGGGLVKPGGSLRLSCAASGFIFNTYTMNWVRQAPGKGLEWVSSITSRGTYIFYSDSLKGRFTISRDNANNSLFLQMNSLRVEDTAVYYCSRDRGYTFGPDYWGQGTLVTVSS

SEQ ID NO: 147 - DNA, 24 bp, synthetic construct
  misc_feature 1..24: note = Synthetic
  source 1..24: mol_type = other DNA
ggattcatcttcaatacctatacc

SEQ ID NO: 148 - protein, 8 aa, synthetic construct
  REGION 1..8: note = Synthetic
GFIFNTYT

SEQ ID NO: 149 - DNA, 24 bp, synthetic construct
  misc_feature 1..24: note = Synthetic
  source 1..24: mol_type = other DNA
atcactagtcgtggtacttatata

SEQ ID NO: 150 - protein, 8 aa, synthetic construct
  REGION 1..8: note = Synthetic
ITSRGTYI

SEQ ID NO: 151 - DNA, 36 bp, synthetic construct
  misc_feature 1..36: note = Synthetic
  source 1..36: mol_type = other DNA
tcgagagatcgtggatacacctttggtcctgactac

SEQ ID NO: 152 - protein, 12 aa, synthetic construct
  REGION 1..12: note = Synthetic
SRDRGYTFGPDY

SEQ ID NO: 153 - DNA, 322 bp, synthetic construct
  misc_feature 1..322: note = Synthetic
  source 1..322: mol_type = other DNA
gacatccagatgacccagtctccatcctccctgtctgcatctgtaggagacagagtcaccatcacttgccgggcaagtcaggacattagaaatgatttaggctggtatcagcagaaaccagggaaagcccctaagggcctgatctatgctgcatccagtttgcaaagtggggtcccatcaaggttcagcggcagtggatctgggacagaattcactctcacaatcagcagcctgcagcctgaagattttgcaacttatcactgtctacattatgattttcatcctcggacgttcggccaagggaccaaggtggaaatcaagc

SEQ ID NO: 154 - protein, 107 aa, synthetic construct
  REGION 1..107: note = Synthetic
DIQMTQSPSSLSASVGDRVTITCRASQDIRNDLGWYQQKPGKAPKGLIYAASSLQSGVPSRFSGSGSGTEFTLTISSLQPEDFATYHCLHYDFHPRTFGQGTKVEIK

SEQ ID NO: 155 - DNA, 18 bp, synthetic construct
  misc_feature 1..18: note = Synthetic
  source 1..18: mol_type = other DNA
caggacattagaaatgat

SEQ ID NO: 156 - protein, 6 aa, synthetic construct
  REGION 1..6: note = Synthetic
QDIRND

SEQ ID NO: 157-158 - skipped (2 entries)

SEQ ID NO: 159 - DNA, 27 bp, synthetic construct
  misc_feature 1..27: note = Synthetic
  source 1..27: mol_type = other DNA
ctacattatgattttcatcctcggacg

SEQ ID NO: 160 - protein, 9 aa, synthetic construct
  REGION 1..9: note = Synthetic
LHYDFHPRT

SEQ ID NO: 161 - DNA, 391 bp, synthetic construct
  misc_feature 1..391: note = Synthetic
  source 1..391: mol_type = other DNA
caggtgcaactggtggagtctgggggaggcgtggtccagcctgggaggtccctgagactctcctgtgcagcgtctggattcaccttcagtagctatgccatgcactgggtccgccaggctccaggcaaggggctggagtgggtggcagttatatggtatgatggaactaattattactatgcagactccgtgaagggccgattcaccatctccagagacaattccaagaacacgctgtatctgcaaatgaacagcctgagagccgaggacacggctgtgtattactgtgcgagagatcccctatattacgatattttgactggttattcccccgactactactacggtatggacgtctggggccaagggaccacggtcaccgtctcctcag

SEQ ID NO: 162 - protein, 130 aa, synthetic construct
  REGION 1..130: note = Synthetic
QVQLVESGGGVVQPGRSLRLSCAASGFTFSSYAMHWVRQAPGKGLEWVAVIWYDGTNYYYADSVKGRFTISRDNSKNTLYLQMNSLRAEDTAVYYCARDPLYYDILTGYSPDYYYGMDVWGQGTTVTVSS

SEQ ID NO: 163 - DNA, 24 bp, synthetic construct
  misc_feature 1..24: note = Synthetic
  source 1..24: mol_type = other DNA
ggattcaccttcagtagctatgcc

SEQ ID NO: 164 - protein, 8 aa, synthetic construct
  REGION 1..8: note = Synthetic
GFTFSSYA

SEQ ID NO: 165 - DNA, 24 bp, synthetic construct
  misc_feature 1..24: note = Synthetic
  source 1..24: mol_type = other DNA
atatggtatgatggaactaattat

SEQ ID NO: 166 - protein, 8 aa, synthetic construct
  REGION 1..8: note = Synthetic
IWYDGTNY

SEQ ID NO: 167 - DNA, 69 bp, synthetic construct
  misc_feature 1..69: note = Synthetic
  source 1..69: mol_type = other DNA
gcgagagatcccctatattacgatattttgactggttattcccccgactactactacggtatggacgtc

SEQ ID NO: 168 - protein, 23 aa, synthetic construct
  REGION 1..23: note = Synthetic
ARDPLYYDILTGYSPDYYYGMDV

SEQ ID NO: 169 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagacttttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggccgctcactttcggcggagggaccaaggtggagatcaaac

SEQ ID NO: 170 - protein, 107 aa, synthetic construct
  REGION 1..107: note = Synthetic
EIVMTQSPATLSVSPGERATLSCRASQTFSSNLAWYQQKPGQAPRLLIYGASTRATGIPARFSGSGSGTEFTLTISSLQSEDFAVYYCQQYNKWPLTFGGGTKVEIK

SEQ ID NO: 171 - DNA, 18 bp, synthetic construct
  misc_feature 1..18: note = Synthetic
  source 1..18: mol_type = other DNA
cagacttttagcagcaac

SEQ ID NO: 172 - protein, 6 aa, synthetic construct
  REGION 1..6: note = Synthetic
QTFSSN

SEQ ID NO: 173-174 - skipped (2 entries)

SEQ ID NO: 175 - DNA, 27 bp, synthetic construct
  misc_feature 1..27: note = Synthetic
  source 1..27: mol_type = other DNA
cagcagtataataagtggccgctcact

SEQ ID NO: 176 - protein, 9 aa, synthetic construct
  REGION 1..9: note = Synthetic
QQYNKWPLT

SEQ ID NO: 177 - DNA, 367 bp, synthetic construct
  misc_feature 1..367: note = Synthetic
  source 1..367: mol_type = other DNA
gaggtgcagctgttggagtctgggggaggcttggtccagcctggggagtccctgagactctcctgtgcagcctctggattcacctttagcagctatgccatgaactgggtccgccaggctccagggaaggggctggagtgggtctcaactatcagtggtagtggtggttatatatactacgcagactccgtgaagggccggttcaccatctccagagacaattccaagaacacgctgtatctgcaaatgaacagcctgagagccgaagacacggccgtatatttctgtgcgaaagattccaggtataactggaactacggcaattttgactactggggccagggaaccctggtcaccgtctcctcag

SEQ ID NO: 178 - protein, 122 aa, synthetic construct
  REGION 1..122: note = Synthetic
EVQLLESGGGLVQPGESLRLSCAASGFTFSSYAMNWVRQAPGKGLEWVSTISGSGGYIYYADSVKGRFTISRDNSKNTLYLQMNSLRAEDTAVYFCAKDSRYNWNYGNFDYWGQGTLVTVSS

SEQ ID NO: 179 - DNA, 24 bp, synthetic construct
  misc_feature 1..24: note = Synthetic
  source 1..24: mol_type = other DNA
ggattcacctttagcagctatgcc

SEQ ID NO: 180 - protein, 8 aa, synthetic construct
  REGION 1..8: note = Synthetic
GFTFSSYA

SEQ ID NO: 181 - DNA, 24 bp, synthetic construct
  misc_feature 1..24: note = Synthetic
  source 1..24: mol_type = other DNA
atcagtggtagtggtggttatata

SEQ ID NO: 182 - protein, 8 aa, synthetic construct
  REGION 1..8: note = Synthetic
ISGSGGYI

SEQ ID NO: 183 - DNA, 45 bp, synthetic construct
  misc_feature 1..45: note = Synthetic
  source 1..45: mol_type = other DNA
gcgaaagattccaggtataactggaactacggcaattttgactac

SEQ ID NO: 184 - protein, 15 aa, synthetic construct
  REGION 1..15: note = Synthetic
AKDSRYNWNYGNFDY

SEQ ID NO: 185 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagaatgttagcagcaacttagcctggaacaagcagaaacctggccaggctcccagactcctcatctatgctacatccaccagggccactggtgtcccagccaggttcagtgccagtgggtctgggacagacttcgctctcaccatcaacagcctgcagtctgaagattttgcagtttattactgtcagcagtataataactggcctctcactttcggcggagggaccaaggtggagatcaaac

SEQ ID NO: 186 - protein, 107 aa, synthetic construct
  REGION 1..107: note = Synthetic
EIVMTQSPATLSVSPGERATLSCRASQNVSSNLAWNKQKPGQAPRLLIYATSTRATGVPARFSASGSGTDFALTINSLQSEDFAVYYCQQYNNWPLTFGGGTKVEIK

SEQ ID NO: 187 - DNA, 18 bp, synthetic construct
  misc_feature 1..18: note = Synthetic
  source 1..18: mol_type = other DNA
cagaatgttagcagcaac

SEQ ID NO: 188 - protein, 6 aa, synthetic construct
  REGION 1..6: note = Synthetic
QNVSSN

SEQ ID NO: 189-190 - skipped (2 entries)

SEQ ID NO: 191 - DNA, 27 bp, synthetic construct
  misc_feature 1..27: note = Synthetic
  source 1..27: mol_type = other DNA
cagcagtataataactggcctctcact

SEQ ID NO: 192 - protein, 9 aa, synthetic construct
  REGION 1..9: note = Synthetic
QQYNNWPLT

SEQ ID NO: 193 - DNA, 367 bp, synthetic construct
  misc_feature 1..367: note = Synthetic
  source 1..367: mol_type = other DNA
gaggtgcaactgttggaatctgggggaggcttggtacagcctggggggtccctgagactctcctgtgtagcctctcgattcaccttcagcagcaatgccatgagttgggtccgccaggctccagggacggggctggagtgggtctcagctattactggtagtggtagtaggacatactacgcagactccgtgaagggccggttcaccatctccagagacaattccaagaacacggtgtatctgcaaatgaacagcctgagaggcgaggacacggccgtatattactgtgcgaaagatcaagggggtacctggaactacggagattttgactactggggccagggaaccctggtcaccgtctcctcag

SEQ ID NO: 194 - protein, 122 aa, synthetic construct
  REGION 1..122: note = Synthetic
EVQLLESGGGLVQPGGSLRLSCVASRFTFSSNAMSWVRQAPGTGLEWVSAITGSGSRTYYADSVKGRFTISRDNSKNTVYLQMNSLRGEDTAVYYCAKDQGGTWNYGDFDYWGQGTLVTVSS

SEQ ID NO: 195 - DNA, 24 bp, synthetic construct
  misc_feature 1..24: note = Synthetic
  source 1..24: mol_type = other DNA
cgattcaccttcagcagcaatgcc

SEQ ID NO: 196 - protein, 8 aa, synthetic construct
  REGION 1..8: note = Synthetic
RFTFSSNA

SEQ ID NO: 197 - DNA, 24 bp, synthetic construct
  misc_feature 1..24: note = Synthetic
  source 1..24: mol_type = other DNA
attactggtagtggtagtaggaca

SEQ ID NO: 198 - protein, 8 aa, synthetic construct
  REGION 1..8: note = Synthetic
ITGSGSRT

SEQ ID NO: 199 - DNA, 45 bp, synthetic construct
  misc_feature 1..45: note = Synthetic
  source 1..45: mol_type = other DNA
gcgaaagatcaagggggtacctggaactacggagattttgactac

SEQ ID NO: 200 - protein, 15 aa, synthetic construct
  REGION 1..15: note = Synthetic
AKDQGGTWNYGDFDY

SEQ ID NO: 201 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtctcccagccaggttcagtggcagtgggtctgggacagacttcactctcaccatcagcagcctgcagtctgaagattttgcagtttattactgtcagcagtataataactggcctctcactttcggcggagggaccaaggtggagatcaagc

SEQ ID NO: 202 - protein, 107 aa, synthetic construct
  REGION 1..107: note = Synthetic
EIVMTQSPATLSVSPGERATLSCRASQSVSSNLAWYQQKPGQAPRLLIYGASTRATGLPARFSGSGSGTDFTLTISSLQSEDFAVYYCQQYNNWPLTFGGGTKVEIK

SEQ ID NO: 203 - DNA, 18 bp, synthetic construct
  misc_feature 1..18: note = Synthetic
  source 1..18: mol_type = other DNA
cagagtgttagcagcaac

SEQ ID NO: 204 - protein, 6 aa, synthetic construct
  REGION 1..6: note = Synthetic
QSVSSN

SEQ ID NO: 205-206 - skipped (2 entries)

SEQ ID NO: 207 - DNA, 27 bp, synthetic construct
  misc_feature 1..27: note = Synthetic
  source 1..27: mol_type = other DNA
cagcagtataataactggcctctcact

SEQ ID NO: 208 - protein, 9 aa, synthetic construct
  REGION 1..9: note = Synthetic
QQYNNWPLT

SEQ ID NO: 209 - DNA, 373 bp, synthetic construct
  misc_feature 1..373: note = Synthetic
  source 1..373: mol_type = other DNA
caggtgcagctggtggagtctgggggagacgtggtccagcctgggaggtccctgagactctcctgtacagcctctggattcaccttcagtagttatggcatgcactgggtccgccaggctccaggcagggggctggagtgggtggcagttatatcatttgatggaaaaaataaatactatgcagactccgtgaagggccgattcaccgtctccagagacaattccaagaacacgctgtttctgcaaatgaacagcctgagagctgaggacacggctctatattactgtgcgaaaaggatagcagcaactggttactactacttctacggtttggacgtctggggccaagggaccacggtcaccgtctcctcag

SEQ ID NO: 210 - protein, 124 aa, synthetic construct
  REGION 1..124: note = Synthetic
QVQLVESGGDVVQPGRSLRLSCTASGFTFSSYGMHWVRQAPGRGLEWVAVISFDGKNKYYADSVKGRFTVSRDNSKNTLFLQMNSLRAEDTALYYCAKRIAATGYYYFYGLDVWGQGTTVTVSS

SEQ ID NO: 211 - DNA, 24 bp, synthetic construct
  misc_feature 1..24: note = Synthetic
  source 1..24: mol_type = other DNA
ggattcaccttcagtagttatggc

SEQ ID NO: 212 - protein, 8 aa, synthetic construct
  REGION 1..8: note = Synthetic
GFTFSSYG

SEQ ID NO: 213 - DNA, 24 bp, synthetic construct
  misc_feature 1..24: note = Synthetic
  source 1..24: mol_type = other DNA
atatcatttgatggaaaaaataaa

SEQ ID NO: 214 - protein, 8 aa, synthetic construct
  REGION 1..8: note = Synthetic
ISFDGKNK

SEQ ID NO: 215 - DNA, 51 bp, synthetic construct
  misc_feature 1..51: note = Synthetic
  source 1..51: mol_type = other DNA
gcgaaaaggatagcagcaactggttactactacttctacggtttggacgtc

SEQ ID NO: 216 - protein, 17 aa, synthetic construct
  REGION 1..17: note = Synthetic
AKRIAATGYYYFYGLDV

SEQ ID NO: 217 - DNA, 322 bp, synthetic construct
  misc_feature 1..322: note = Synthetic
  source 1..322: mol_type = other DNA
gaaataatgatgacgcagtctccagccaccctgtctgtgtctccaggggaaagaggcaccctctcctgcagggccagtcagagtgttagtagcaacttagcctggtaccagcagaaacctggccaggctcccaggctcctcatctatggtgcatccaccagggccagtggtatcccagccaggttcagtggcagtgggtctgggacagagttcactctcaccgtcagcagcctgcagtctgaagattttgcagtttattactgtcagcagtataataactggccgctcactttcggcggagggaccaaggtggagatcaaac

SEQ ID NO: 218 - protein, 107 aa, synthetic construct
  REGION 1..107: note = Synthetic
EIMMTQSPATLSVSPGERGTLSCRASQSVSSNLAWYQQKPGQAPRLLIYGASTRASGIPARFSGSGSGTEFTLTVSSLQSEDFAVYYCQQYNNWPLTFGGGTKVEIK

SEQ ID NO: 219 - DNA, 18 bp, synthetic construct
  misc_feature 1..18: note = Synthetic
  source 1..18: mol_type = other DNA
cagagtgttagtagcaac

SEQ ID NO: 220 - protein, 6 aa, synthetic construct
  REGION 1..6: note = Synthetic
QSVSSN

SEQ ID NO: 221-222 - skipped (2 entries)

SEQ ID NO: 223 - DNA, 27 bp, synthetic construct
  misc_feature 1..27: note = Synthetic
  source 1..27: mol_type = other DNA
cagcagtataataactggccgctcact

SEQ ID NO: 224 - protein, 9 aa, synthetic construct
  REGION 1..9: note = Synthetic
QQYNNWPLT

SEQ ID NO: 225 - DNA, 367 bp, synthetic construct
  misc_feature 1..367: note = Synthetic
  source 1..367: mol_type = other DNA
gaggttcagctgttggagtctgggggaggcttggtacagccgggggggtccctgagactctcctgtgcagcctctggattcacctctatcacctatgccatgagctgggtccgccaggctccagggaaggggctggagtgggtctcagctattagtgttagtggtactaatacatactacgcagactccgtgaagggccggttcaccatctccagagacaagtccaagaacatgctgtatctgcaaatgaacagcctgagagccgaggacacggccgtgtattactgtgcgaaagatctcctacataactggaaatacgggacttttgatatctggggccaagggacaatggtcaccgtctcttcag

SEQ ID NO: 226 - protein, 122 aa, synthetic construct
  REGION 1..122: note = Synthetic
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 227 - DNA, 24 bp, synthetic construct
  misc_feature 1..24: note = Synthetic
  source 1..24: mol_type = other DNA
ggattcacctctatcacctatgcc

SEQ ID NO: 228 - protein, 8 aa, synthetic construct
  REGION 1..8: note = Synthetic
GFTSITYA

SEQ ID NO: 229 - DNA, 24 bp, synthetic construct
  misc_feature 1..24: note = Synthetic
  source 1..24: mol_type = other DNA
attagtgttagtggtactaataca

SEQ ID NO: 230 - protein, 8 aa, synthetic construct
  REGION 1..8: note = Synthetic
ISVSGTNT

SEQ ID NO: 231 - DNA, 45 bp, synthetic construct
  misc_feature 1..45: note = Synthetic
  source 1..45: mol_type = other DNA
gcgaaagatctcctacataactggaaatacgggacttttgatatc

SEQ ID NO: 232 - protein, 15 aa, synthetic construct
  REGION 1..15: note = Synthetic
AKDLLHNWKYGTFDI

SEQ ID NO: 233 - DNA, 322 bp, synthetic construct
  misc_feature 1..322: note = Synthetic
  source 1..322: mol_type = other DNA
gaaatagtgatgacgcagtctccagccaccctgtctgtgtctccaggggaaagagccaccctctcctgcagggccagtcagagtgttgacagcaacttagtctggtaccaacaaaaacctggccaggttcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggccgctcactttcggcggagggaccaaggtggagatcaaac

SEQ ID NO: 234 - protein, 107 aa, synthetic construct
  REGION 1..107: note = Synthetic
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 235 - DNA, 18 bp, synthetic construct
  misc_feature 1..18: note = Synthetic
  source 1..18: mol_type = other DNA
cagagtgttgacagcaac

SEQ ID NO: 236 - protein, 6 aa, synthetic construct
  REGION 1..6: note = Synthetic
QSVDSN

SEQ ID NO: 237-238 - skipped (2 entries)

SEQ ID NO: 239 - DNA, 27 bp, synthetic construct
  misc_feature 1..27: note = Synthetic
  source 1..27: mol_type = other DNA
cagcagtataataagtggccgctcact

SEQ ID NO: 240 - protein, 9 aa, synthetic construct
  REGION 1..9: note = Synthetic
QQYNKWPLT

SEQ ID NO: 241 - DNA, 367 bp, synthetic construct; identical to SEQ ID NO: 225
  misc_feature 1..367: note = Synthetic
  source 1..367: mol_type = other DNA

SEQ ID NO: 242 - protein, 122 aa, synthetic construct; identical to SEQ ID NO: 226
  REGION 1..122: note = Synthetic

SEQ ID NO: 243 - DNA, 24 bp, synthetic construct
  misc_feature 1..24: note = Synthetic
  source 1..24: mol_type = other DNA
ggattcacctctatcacctatgcc

SEQ ID NO: 244 - protein, 8 aa, synthetic construct
  REGION 1..8: note = Synthetic
GFTSITYA

SEQ ID NO: 245 - DNA, 24 bp, synthetic construct
  misc_feature 1..24: note = Synthetic
  source 1..24: mol_type = other DNA
attagtgttagtggtactaataca

SEQ ID NO: 246 - protein, 8 aa, synthetic construct
  REGION 1..8: note = Synthetic
ISVSGTNT

SEQ ID NO: 247 - DNA, 45 bp, synthetic construct; identical to SEQ ID NO: 231
  misc_feature 1..45: note = Synthetic
  source 1..45: mol_type = other DNA

SEQ ID NO: 248 - protein, 15 aa, synthetic construct
  REGION 1..15: note = Synthetic
AKDLLHNWKYGTFDI

SEQ ID NO: 249 - DNA, 325 bp, synthetic construct
  misc_feature 1..325: note = Synthetic
  source 1..325: mol_type = other DNA
gaaatagtgatgacgcagtctccagccaccctgtctgtgtctccaggggaaagagccaccctctcctgcagggccagtcagagtgttagcagcaacttagcctggaaccagcagaaacctggccaggctcccaggctcctcatctatggtgcatccaccagggccacgggtatcccagccaggttcagtggcagtgggtctgggacagagttcactctcaccatcagcagcctgcagtctgaagattttgcagtttattactgtcagcagtataacaactggcccatgtacacttttggccaggggaccaagctggagatcaaac

SEQ ID NO: 250 - protein, 108 aa, synthetic construct
  REGION 1..108: note = Synthetic
EIVMTQSPATLSVSPGERATLSCRASQSVSSNLAWNQQKPGQAPRLLIYGASTRATGIPARFSGSGSGTEFTLTISSLQSEDFAVYYCQQYNNWPMYTFGQGTKLEIK

SEQ ID NO: 251 - DNA, 18 bp, synthetic construct
  misc_feature 1..18: note = Synthetic
  source 1..18: mol_type = other DNA
cagagtgttagcagcaac

SEQ ID NO: 252 - protein, 6 aa, synthetic construct
  REGION 1..6: note = Synthetic
QSVSSN

SEQ ID NO: 253-254 - skipped (2 entries)

SEQ ID NO: 255 - DNA, 30 bp, synthetic construct
  misc_feature 1..30: note = Synthetic
  source 1..30: mol_type = other DNA
cagcagtataacaactggcccatgtacact

SEQ ID NO: 256 - protein, 10 aa, synthetic construct
  REGION 1..10: note = Synthetic
QQYNNWPMYT

SEQ ID NO: 257 - DNA, 361 bp, synthetic construct
  misc_feature 1..361: note = Synthetic
  source 1..361: mol_type = other DNA
caggtgcagctggtggagtctgggggaggcgtggtccagcctgggaggtccctgagactctcctgtgaagcctctggattcaccttcagtagttctggcatgcactgggtccgccaggctccaggcaaggggctgcagtgggtggctgttatatcatatgatggaaataataaattttatgaagactccgtgaagggccgattgaccatttccagagacaattccaacaacactctgtggctgcaaatgaacagcctgagagttgaagacacggctgtttattactgtgcgaaatcaggaggtagagtgggagccgcctttgcctactggggccagggaaccctggtcaccgtctcctcag

SEQ ID NO: 258 - protein, 120 aa, synthetic construct
  REGION 1..120: note = Synthetic
QVQLVESGGGVVQPGRSLRLSCEASGFTFSSSGMHWVRQAPGKGLQWVAVISYDGNNKFYEDSVKGRLTISRDNSNNTLWLQMNSLRVEDTAVYYCAKSGGRVGAAFAYWGQGTLVTVSS

SEQ ID NO: 259 - DNA, 24 bp, synthetic construct
  misc_feature 1..24: note = Synthetic
  source 1..24: mol_type = other DNA
ggattcaccttcagtagttctggc

SEQ ID NO: 260 - protein, 8 aa, synthetic construct
  REGION 1..8: note = Synthetic
GFTFSSSG

SEQ ID NO: 261 - DNA, 24 bp, synthetic construct
  misc_feature 1..24: note = Synthetic
  source 1..24: mol_type = other DNA
atatcatatgatggaaataataaa

SEQ ID NO: 262 - protein, 8 aa, synthetic construct
  REGION 1..8: note = Synthetic
ISYDGNNK

SEQ ID NO: 263 - DNA, 39 bp, synthetic construct
  misc_feature 1..39: note = Synthetic
  source 1..39: mol_type = other DNA
gcgaaatcaggaggtagagtgggagccgcctttgcctac

SEQ ID NO: 264 - protein, 13 aa, synthetic construct
  REGION 1..13: note = Synthetic
AKSGGRVGAAFAY

SEQ ID NO: 265 - DNA, 337 bp, synthetic construct
  misc_feature 1..337: note = Synthetic
  source 1..337: mol_type = other DNA
gatattgtgaacactcagtctccactctctctgcccgtcacccctggagagccggcctccatctcctgcaggtctagtcagagcctcctgtatggtaatggatacaactatttggattggtacctgcagaagccagggcactctccacagctcctgatctatttgggttctaatcggggctccggggtccctgacaggttcagtggcagtggatcaggcacagattttacactgaagatcagcagagtggaggctgaagatgttggcatttattactgcatgcaaactctacaaactccattcactttcggccctgggaccaaaatgtatatcaaac

SEQ ID NO: 266 - protein, 112 aa, synthetic construct
  REGION 1..112: note = Synthetic
DIVNTQSPLSLPVTPGEPASISCRSSQSLLYGNGYNYLDWYLQKPGHSPQLLIYLGSNRGSGVPDRFSGSGSGTDFTLKISRVEAEDVGIYYCMQTLQTPFTFGPGTKMYIK

SEQ ID NO: 267 - DNA, 33 bp, synthetic construct
  misc_feature 1..33: note = Synthetic
  source 1..33: mol_type = other DNA
cagagcctcctgtatggtaatggatacaactat

SEQ ID NO: 268 - protein, 11 aa, synthetic construct
  REGION 1..11: note = Synthetic
QSLLYGNGYNY

SEQ ID NO: 269-270 - skipped (2 entries)

SEQ ID NO: 271 - DNA, 27 bp, synthetic construct
  misc_feature 1..27: note = Synthetic
  source 1..27: mol_type = other DNA
atgcaaactctacaaactccattcact

SEQ ID NO: 272 - protein, 9 aa, synthetic construct
  REGION 1..9: note = Synthetic
MQTLQTPFT

SEQ ID NO: 273 - DNA, 376 bp, synthetic construct
  misc_feature 1..376: note = Synthetic
  source 1..376: mol_type = other DNA
gaggtgcagctgttggagtctgggggaggcttggtacagcctggggggtccctgagactctcctgtgcaggctctggaatcacctttagcagctatgccatgagctgggtccgccaggctccagggaaggggctggagtgggtctcagctattagtggtaatggtggtaccacaaactacgcagactccgtgaagggccggttcaccatctccagagacaactccaagaacacgctgtatctgcaaatgaacagcctgagagccgaggacacggccgtatattactgtgcgaaagaaagaatccttaccagcagctggacgaggtacggtattatggacgtctggggccaagggaccacggtcaccgtctcctcag

SEQ ID NO: 274 - protein, 125 aa, synthetic construct
  REGION 1..125: note = Synthetic
EVQLLESGGGLVQPGGSLRLSCAGSGITFSSYAMSWVRQAPGKGLEWVSAISGNGGTTNYADSVKGRFTISRDNSKNTLYLQMNSLRAEDTAVYYCAKERILTSSWTRYGIMDVWGQGTTVTVSS

SEQ ID NO: 275 - DNA, 24 bp, synthetic construct
  misc_feature 1..24: note = Synthetic
  source 1..24: mol_type = other DNA
ggaatcacctttagcagctatgcc

SEQ ID NO: 276 - protein, 8 aa, synthetic construct
  REGION 1..8: note = Synthetic
GITFSSYA

SEQ ID NO: 277 - DNA, 24 bp, synthetic construct
  misc_feature 1..24: note = Synthetic
  source 1..24: mol_type = other DNA
attagtggtaatggtggtaccaca

SEQ ID NO: 278 - protein, 8 aa, synthetic construct
  REGION 1..8: note = Synthetic
ISGNGGTT

SEQ ID NO: 279 - DNA, 54 bp, synthetic construct
  misc_feature 1..54: note = Synthetic
  source 1..54: mol_type = other DNA
gcgaaagaaagaatccttaccagcagctggacgaggtacggtattatggacgtc

SEQ ID NO: 280 - protein, 18 aa, synthetic construct
  REGION 1..18: note = Synthetic
AKERILTSSWTRYGIMDV

SEQ ID NO: 281 - DNA, 322 bp, synthetic construct
  misc_feature 1..322: note = Synthetic
  source 1..322: mol_type = other DNA
gaaatagtgatgacgcagtctccagccaccctgtctatgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaagttagagatcaaac

SEQ ID NO: 282 - protein, 107 aa, synthetic construct
  REGION 1..107: note = Synthetic
EIVMTQSPATLSMSPGERATLSCRASQSVSSNLAWYQQKPGQAPRLLIYGASTRATGIPARFSGSGSGTEFTLTISSLQSEDFAVYYCQQYNNWPLTFGGGTKLEIK

SEQ ID NO: 283 - DNA, 18 bp, synthetic construct
  misc_feature 1..18: note = Synthetic
  source 1..18: mol_type = other DNA
cagagtgttagcagcaac

SEQ ID NO: 284 - protein, 6 aa, synthetic construct
  REGION 1..6: note = Synthetic
QSVSSN

SEQ ID NO: 285-286 - skipped (2 entries)

SEQ ID NO: 287 - DNA, 27 bp, synthetic construct
  misc_feature 1..27: note = Synthetic
  source 1..27: mol_type = other DNA
cagcagtataataactggcctctcact

SEQ ID NO: 288 - protein, 9 aa, synthetic construct
  REGION 1..9: note = Synthetic
QQYNNWPLT

SEQ ID NO: 289 - DNA, 358 bp, synthetic construct
  misc_feature 1..358: note = Synthetic
  source 1..358: mol_type = other DNA
caggtgcaactacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaaacaccaactacaacccgtccctcaagagtcgagtcaccatatcagtagacacgtccaagaaccagttctccctgaaactgaactctgtgaccgccgcggacacggctgtgtattactgtgcgagaagagaggctacagtaactccatactttgactactggggccagggaaccctggtcaccgtctcctcag

SEQ ID NO: 290 - protein, 119 aa, synthetic construct
  REGION 1..119: note = Synthetic
QVQLQQWGAGLLKPSETLSLTCAVYGGSFSGYYWSWIRQPPGKGLEWIGEINHSGNTNYNPSLKSRVTISVDTSKNQFSLKLNSVTAADTAVYYCARREATVTPYFDYWGQGTLVTVSS

SEQ ID NO: 291 - DNA, 24 bp, synthetic construct
  misc_feature 1..24: note = Synthetic
  source 1..24: mol_type = other DNA
ggtgggtccttcagtggttactac

SEQ ID NO: 292 - protein, 8 aa, synthetic construct
  REGION 1..8: note = Synthetic
GGSGSGYY

SEQ ID NO: 293 - DNA, 21 bp, synthetic construct
  misc_feature 1..21: note = Synthetic
  source 1..21: mol_type = other DNA
atcaatcatagtggaaacacc

SEQ ID NO: 294 - protein, 7 aa, synthetic construct
  REGION 1..7: note = Synthetic
INHSGNT

SEQ ID NO: 295 - DNA, 39 bp, synthetic construct
  misc_feature 1..39: note = Synthetic
  source 1..39: mol_type = other DNA
gcgagaagagaggctacagtaactccatactttgactac

SEQ ID NO: 296 - protein, 13 aa, synthetic construct
  REGION 1..13: note = Synthetic
ARREATVTPYFDY

SEQ ID NO: 297 - DNA, 322 bp, synthetic construct
  misc_feature 1..322: note = Synthetic
  source 1..322: mol_type = other DNA
gacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttatccgctcactttcggcggagggaccaaggtggagatcaaac

SEQ ID NO: 298 - protein, 107 aa, synthetic construct
  REGION 1..107: note = Synthetic
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 299 - DNA, 18 bp, synthetic construct
  misc_feature 1..18: note = Synthetic
  source 1..18: mol_type = other DNA
cagggcattagcagttat

SEQ ID NO: 300 - protein, 6 aa, synthetic construct
  REGION 1..6: note = Synthetic
QGISSY

SEQ ID NO: 301-302 - skipped (2 entries)

SEQ ID NO: 303 - DNA, 27 bp, synthetic construct
  misc_feature 1..27: note = Synthetic
  source 1..27: mol_type = other DNA
caacagcttaatagttatccgctcact

SEQ ID NO: 304 - protein, 9 aa, synthetic construct
  REGION 1..9: note = Synthetic
QQLNSYPLT

SEQ ID NO: 305 - DNA, 355 bp, synthetic construct
  misc_feature 1..355: note = Synthetic
  source 1..355: mol_type = other DNA
caggtgcagctggtggagtctgggggaggcgtggtccagcctgggaggtccctgagactctcctgtgtagtctctggattcaacttcagtaggaatggcatacactgggtccgccaggctccaggcaaggggctggaatgggtggcagttatatcatatgatggaagaaataaattttatgtagagtccgtgaagggccgattcaccatctccagagacaattccaagaacacgctgtatctgcaaatgaacagtctgagagttgaggacacggctgtatattactgtgcgaaatcctcaattggagggttttttgaatactggggccagggaaccctggtcaccgtctcctcag

SEQ ID NO: 306 - protein, 118 aa, synthetic construct
  REGION 1..118: note = Synthetic
QVQLVESGGGVVQPGRSLRLSCVVSGFNFSRNGIHWVRQAPGKGLEWVAVISYDGRNKFYVESVKGRFTISRDNSKNTLYLQMNSLRVEDTAVYYCAKSSIGGFFEYWGQGTLVTVSS

SEQ ID NO: 307 - DNA, 24 bp, synthetic construct
  misc_feature 1..24: note = Synthetic
  source 1..24: mol_type = other DNA
ggattcaacttcagtaggaatggc

SEQ ID NO: 308 - protein, 8 aa, synthetic construct
  REGION 1..8: note = Synthetic
GFNFSRNG

SEQ ID NO: 309 - DNA, 24 bp, synthetic construct
  misc_feature 1..24: note = Synthetic
  source 1..24: mol_type = other DNA
atatcatatgatggaagaaataaa

SEQ ID NO: 310 - protein, 8 aa, synthetic construct
  REGION 1..8: note = Synthetic
ISYDGRNK

SEQ ID NO: 311 - DNA, 33 bp, synthetic construct
  misc_feature 1..33: note = Synthetic
  source 1..33: mol_type = other DNA
gcgaaatcctcaattggagggttttttgaatac

SEQ ID NO: 312 - protein, 11 aa, synthetic construct
  REGION 1..11: note = Synthetic
AKSSIGGFFEY

SEQ ID NO: 313 - DNA, 337 bp, synthetic construct
  misc_feature 1..337: note = Synthetic
  source 1..337: mol_type = other DNA
gatattgtgatgactcagtctccactctccctgcccgtcactcctggagagccggcctccatctcctgcaggtctagtcagagcctcctgcatagtaatggatacaactatttggattggtacctgcagaagccaggacagtctccacaactcatgatctatttgggttctcatcgggcctccggggtccctgacaggttcagtggcagtggatcaggcacagattttacactgaaaatcagcagagtggaggctgaggatgttggagtctattactgcattcaagttcaacaaactccgatcaccttcggccaagggacacggctggagattaaac

SEQ ID NO: 314 - protein, 112 aa, synthetic construct
  REGION 1..112: note = Synthetic
DIVMTQSPLSLPVTPGEPASISCRSSQSLLHSNGYNYLDWYLQKPGQSPQLMIYLGSHRASGVPDRFSGSGSGTDFTLKISRVEAEDVGVYYCIQVQQTPITFGQGTRLEIK

SEQ ID NO: 315 - DNA, 33 bp, synthetic construct
  misc_feature 1..33: note = Synthetic
  source 1..33: mol_type = other DNA
cagagcctcctgcatagtaatggatacaactat

SEQ ID NO: 316 - protein, 11 aa, synthetic construct
  REGION 1..11: note = Synthetic
QSLLHSNGYNY

SEQ ID NO: 317-318 - skipped (2 entries)

SEQ ID NO: 319 - DNA, 27 bp, synthetic construct
  misc_feature 1..27: note = Synthetic
  source 1..27: mol_type = other DNA
attcaagttcaacaaactccgatcacc

SEQ ID NO: 320 - protein, 9 aa, synthetic construct
  REGION 1..9: note = Synthetic
IQVQQTPIT

SEQ ID NO: 321 - DNA, 337 bp, synthetic construct
  misc_feature 1..337: note = Synthetic
  source 1..337: mol_type = other DNA
gatattgtgatgactcagactccactctcctcacctgtcacccttggacagccggcctccatctcctgcaggtctagtcaaagcctcgtacacagtgatggaaacacctacttgagttggcttcagcagaggccaggccagcctccaagactcctaatttacaagatttctaaccggttctctggggtcccagacagattcagtggcagtggggcagggacagatttcacactgaaaatcagcagggtggaagctgaggatgtcggggtttatttctgcatgcaagctacacaatttccgtacacttttggccaggggaccaagctggagatcaaag

SEQ ID NO: 322 - protein, 112 aa, synthetic construct
  REGION 1..112: note = Synthetic
DIVMTQTPLSSPVTLGQPASISCRSSQSLVHSDGNTYLSWLQQRPGQPPRLLIYKISNRFSGVPDRFSGSGAGTDFTLKISRVEAEDVGVYFCMQATQFPYTFGQGTKLEIK

SEQ ID NO: 323 - DNA, 33 bp, synthetic construct
  misc_feature 1..33: note = Synthetic
  source 1..33: mol_type = other DNA
caaagcctcgtacacagtgatggaaacacctac

SEQ ID NO: 324 - protein, 11 aa, synthetic construct
  REGION 1..11: note = Synthetic
QSLVHSDGNTY

SEQ ID NO: 325-326 - skipped (2 entries)

SEQ ID NO: 327 - DNA, 27 bp, synthetic construct
  misc_feature 1..27: note = Synthetic
  source 1..27: mol_type = other DNA
atgcaagctacacaatttccgtacact

SEQ ID NO: 328 - protein, 9 aa, synthetic construct
  REGION 1..9: note = Synthetic
MQATQFPYT

SEQ ID NO: 329-334 - skipped (6 entries)

SEQ ID NO: 335 - protein, 330 aa, synthetic construct [sequence omitted]

SEQ ID NO: 336 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 337 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 338 - DNA, 1128 bp, Homo sapiens
  source 1..1128: mol_type = other DNA
atgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcatga

SEQ ID NO: 339 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 340 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 341 - DNA, 1224 bp, Homo sapiens
  source 1..1224: mol_type = other DNA
atggtgctcgcggccccgctgctgctgggcttcctgctcctcgccctggagctgcggccccggggggaggcggccgagggccccgcggcggcggcggcggcggcggcggcggcggcagcggcgggggtcgggggggagcgctccagccggccagccccgtccgtggcgcccgagccggacggctgccccgtgtgcgtttggcggcagcacagccgcgagctgcgcctagagagcatcaagtcgcagatcttgagcaaactgcggctcaaggaggcgcccaacatcagccgcgaggtggtgaagcagctgctgcccaaggcgccgccgctgcagcagatcctggacctacacgacttccagggcgacgcgctgcagcccgaggacttcctggaggaggacgagtaccacgccaccaccgagaccgtcattagcatggcccaggagacggacccagcagtacagacagatggcagccctctctgctgccattttcacttcagccccaaggtgatgttcacaaaggtactgaaggcccagctgtgggtgtacctacggcctgtaccccgcccagccacagtctacctgcagatcttgcgactaaaacccctaactggggaagggaccgcagggggagggggcggaggccggcgtcacatccgtatccgctcactgaagattgagctgcactcacgctcaggccattggcagagcatcgacttcaagcaagtgctacacagctggttccgccagccacagagcaactggggcatcgagatcaacgcctttgatcccagtggcacagacctggctgtcacctccctggggccgggagccgaggggctgcatccattcatggagcttcgagtcctagagaacacaaaacgttcccggcggaacctgggtctggactgcgacgagcactcaagcgagtcccgctgctgccgatatcccctcacagtggactttgaggctttcggctgggactggatcatcgcacctaagcgctacaaggccaactactgctccggccagtgcgagtacatgttcatgcaaaaatatccgcatacccatttggtgcagcaggccaatccaagaggctctgctgggccctgttgtacccccaccaagatgtccccaatcaacatgctctacttcaatgacaagcagcagattatctacggcaagatccctggcatggtggtggatcgctgtggctgctcttaa

SEQ ID NO: 342 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 343 - protein, 109 aa, Homo sapiens
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 344 - DNA, 1503 bp, Homo sapiens
  source 1..1503: mol_type = other DNA
atgagactccccaaactcctcactttcttgctttggtacctggcttggctggacctggaattcatctgcactgtgttgggtgcccctgacttgggccagagaccccaggggaccaggccaggattggccaaagcagaggccaaggagaggccccccctggcccggaacgtcttcaggccagggggtcacagctatggtgggggggccaccaatgccaatgccagggcaaagggaggcaccgggcagacaggaggcctgacacagcccaagaaggatgaacccaaaaagctgccccccagaccgggcggccctgaacccaagccaggacaccctccccaaacaaggcaggctacagcccggactgtgaccccaaaaggacagcttcccggaggcaaggcacccccaaaagcaggatctgtccccagctccttcctgctgaagaaggccagggagcccgggcccccacgagagcccaaggagccgtttcgcccaccccccatcacaccccacgagtacatgctctcgctgtacaggacgctgtccgatgctgacagaaagggaggcaacagcagcgtgaagttggaggctggcctggccaacaccatcaccagctttattgacaaagggcaagatgaccgaggtcccgtggtcaggaagcagaggtacgtgtttgacattagtgccctggagaaggatgggctgctgggggccgagctgcggatcttgcggaagaagccctcggacacggccaagccagcggcccccggaggcgggcgggctgcccagctgaagctgtccagctgccccagcggccggcagccggcctccttgctggatgtgcgctccgtgccaggcctggacggatctggctgggaggtgttcgacatctggaagctcttccgaaactttaagaactcggcccagctgtgcctggagctggaggcctgggaacggggcagggccgtggacctccgtggcctgggcttcgaccgcgccgcccggcaggtccacgagaaggccctgttcctggtgtttggccgcaccaagaaacgggacctgttctttaatgagattaaggcccgctctggccaggacgataagaccgtgtatgagtacctgttcagccagcggcgaaaacggcgggccccactggccactcgccagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccacctgctgtgtgcccacgcggctgagtcccatcagcatcctcttcattgactctgccaacaacgtggtgtataagcagtatgaggacatggtcgtggagtcgtgtggctgcagg

SEQ ID NO: 345 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 346 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 347 - protein, 107 aa, synthetic construct
  REGION 1..107: note = Synthetic
QAKHKQRKRLKSSCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 348 - protein, 109 aa, synthetic construct
  REGION 1..109: note = Synthetic
ALDTNYCFSSTEKNCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 349 - protein, 111 aa, synthetic construct
  REGION 1..111: note = Synthetic
DFGLDCDEHSTESRCCKRHPLYVDFSDVGWNDWIVAPPGYHAFYCSGECEFVFLQKYPHTHLVHQANPRGSAGPCCTPTKMSPINMLYFNGKEQIIYGKIPAMVVDRCGCS

SEQ ID NO: 350 - protein, 110 aa, synthetic construct
  REGION 1..110: note = Synthetic
DFGLDCDEHSTESRCCVRQLYIDFRKDLGWKWIHEPKGYHANFCSGECEFVFLQKYPHTHLVHQANPRGSAGPCCTPTKMSPINMLYFNGKEQIIYGKIPAMVVDRCGCS

SEQ ID NO: 351 - protein, 114 aa, synthetic construct
  REGION 1..114: note = Synthetic
DFGLDCDEHSTESRCCRYPLTVDFEAFGWDWIIAPKRYKANYCSGECPFPLADHLNSTNHAIVQTLVNSVNSKIPKACCTPTKMSPINMLYFNGKEQIIYGKIPAMVVDRCGCS

SEQ ID NO: 352 - protein, 112 aa, synthetic construct
  REGION 1..112: note = Synthetic
DFGLDCDEHSTESRCCRYPLTVDFEAFGWDWIIAPKRYKANYCSGECPYIWSLDTQYSKVLALYNQHNPGASAAPCCTPTKMSPINMLYFNGKEQIIYGKIPAMVVDRCGCS

SEQ ID NO: 353 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANSVNSKIPCCTPTKMSPINMLYFNGKEQIIYGKIPAMVVDRCGCS

SEQ ID NO: 354 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPGASAAPCCTPTKMSPINMLYFNGKEQIIYGKIPAMVVDRCGCS

SEQ ID NO: 355 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPRGSAGPCCVPTELSAISMLYLDENEKVVLKNYQDMVVEGCGCS

SEQ ID NO: 356 - protein, 108 aa, synthetic construct
  REGION 1..108: note = Synthetic
DFGLDCDEHSTESRCCRYPLTVDFEAFGWDWIIAPKRYKANYCSGECEFVFLQKYPHTHLVHQANPRGSAGPCCVPQALEPLPIVYYVGRKPKVEQLSNMIVRSCGCS

SEQ ID NO: 357 - protein, 109 aa, synthetic construct
  REGION 1..109: note = Synthetic
DFGLDCDEHSTESRCCRYPLTVDFEAFGWDWIIAPKRYKANYCSGECEFVFLQKYPHTHLVHQANPRGSAGPCCTPTKMSPINMLYFNGKEKVVLKNYQDMVVEGCGCS

SEQ ID NO: 358 - protein, 108 aa, synthetic construct
  REGION 1..108: note = Synthetic
DFGLDCDEHSTESRCCRYPLTVDFEAFGWDWIIAPKRYKANYCSGECEFVFLQKYPHTHLVHQANPRGSAGPCCTPTKMSPINMLYFNGKKPKVEQLSNMIVRSCGCS

SEQ ID NO: 359 - DNA, 360 bp, synthetic construct
  misc_feature 1..360: note = Synthetic
  source 1..360: mol_type = other DNA
gaggtgcaggtgttggagtctgggggagacttggtacagcctggggggtccctgagactctcctgtgcagcctctggattcacctttagtgcctatgccatgacctgggtccgccaggctccagggaaggggctggagtgggtctcagctattagtggtagtggtggtagcgcatactacgcagactccgtgaagggccggttcaccatctccagagacaattccaagaacacggtatatctgcagatgaacagcctgagagccgaggacacggccgtatattactgtgcgaaagatggggcctggaaaatgtccggtttggacgtctggggccaagggaccacggtcatcgtctcctca

SEQ ID NO: 360 - protein, 120 aa, synthetic construct
  REGION 1..120: note = Synthetic
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 361 - DNA, 24 bp, synthetic construct
  misc_feature 1..24: note = Synthetic
  source 1..24: mol_type = other DNA
ggattcacctttagtgcctatgcc

SEQ ID NO: 362 - protein, 8 aa, synthetic construct
  REGION 1..8: note = Synthetic
GFTFSAYA

SEQ ID NO: 363 - DNA, 24 bp, synthetic construct
  misc_feature 1..24: note = Synthetic
  source 1..24: mol_type = other DNA
attagtggtagtggtggtagcgca

SEQ ID NO: 364 - protein, 8 aa, synthetic construct
  REGION 1..8: note = Synthetic
ISGSGGSA

SEQ ID NO: 365 - DNA, 39 bp, synthetic construct
  misc_feature 1..39: note = Synthetic
  source 1..39: mol_type = other DNA
gcgaaagatggggcctggaaaatgtccggtttggacgtc

SEQ ID NO: 366 - protein, 13 aa, synthetic construct
  REGION 1..13: note = Synthetic
AKDGAWKMSGLDV

SEQ ID NO: 367 - DNA, 321 bp, synthetic construct
  misc_feature 1..321: note = Synthetic
  source 1..321: mol_type = other DNA
gacatccagatgacccagtctccagcctccctgtctgcatctgttggagacagagtcaccatcacttgtcgggcgagtcaggacattagcgattatttagcctggtatcagcagaaaccagggaaaattcctaggctcctgatctatactacatccactttgcaatcaggggtcccatctcggttccgtggcagtgggtctgggacagatttcactctcaccatcagcagcctgcagcctgaagatgttgcaacttattactgtcagaagtatgacagtgccccgctcactttcggcggagggaccaaggtggagatcaaa

SEQ ID NO: 368 - protein, 107 aa, synthetic construct
  REGION 1..107: note = Synthetic
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 369 - DNA, 18 bp, synthetic construct
  misc_feature 1..18: note = Synthetic
  source 1..18: mol_type = other DNA
caggacattagcgattat

SEQ ID NO: 370 - protein, 6 aa, synthetic construct
  REGION 1..6: note = Synthetic
QDISDY

SEQ ID NO: 371-372 - skipped (2 entries)

SEQ ID NO: 373 - DNA, 27 bp, synthetic construct
  misc_feature 1..27: note = Synthetic
  source 1..27: mol_type = other DNA
cagaagtatgacagtgccccgctcact

SEQ ID NO: 374 - protein, 9 aa, synthetic construct
  REGION 1..9: note = Synthetic
QKYDSAPLT

SEQ ID NO: 375 - DNA, 363 bp, synthetic construct
  misc_feature 1..363: note = Synthetic
  source 1..363: mol_type = other DNA
caggtgcagctggtggagtctgggggaggcgtggtccagcctgggaagtccctgcgactctcctgtgcagcgtctggattcaccttcagtagttttggcatgcattgggtccgccaggctccaggcaaggggctggagtgggtggcagttattgggtatgatggaggtaatgaatactatgccgactccgtgaagggccgattcaccatctccagagacaattccaagaacacgctgaatctgcaaatgagcagcctgagagccgaagacacggctgtgtattattgttcgactataagtcattacgatattttgagcggtatggacgtctggggccgagggaccacggtcaccgtctcctca

SEQ ID NO: 376 - protein, 121 aa, synthetic construct
  REGION 1..121: note = Synthetic
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 377 - DNA, 24 bp, synthetic construct
  misc_feature 1..24: note = Synthetic
  source 1..24: mol_type = other DNA
ggattcaccttcagtagttttggc

SEQ ID NO: 378 - protein, 8 aa, synthetic construct
  REGION 1..8: note = Synthetic
GFTFSSFG

SEQ ID NO: 379 - DNA, 24 bp, synthetic construct
  misc_feature 1..24: note = Synthetic
  source 1..24: mol_type = other DNA
attgggtatgatggaggtaatgaa

SEQ ID NO: 380 - protein, 8 aa, synthetic construct
  REGION 1..8: note = Synthetic
IGYDGGNE

SEQ ID NO: 381 - DNA, 42 bp, synthetic construct
  misc_feature 1..42: note = Synthetic
  source 1..42: mol_type = other DNA
tcgactataagtcattacgatattttgagcggtatggacgtc

SEQ ID NO: 382 - protein, 14 aa, synthetic construct
  REGION 1..14: note = Synthetic
STISHYDILSGMDV

SEQ ID NO: 383 - DNA, 324 bp, synthetic construct
  misc_feature 1..324: note = Synthetic
  source 1..324: mol_type = other DNA
gacatccagatgacccagtctccatcttccgtgtctgcatctgtaggagacagagtcaccatcacttgtcgggcgagtcagggtattagcaactggttagcctggtatcagcagaaaccagggaaagcccctaagctcctgatctttgctgcatccagtttgcaaagtggggtcccatcacggttcagcggcagtgcatctgggacagatttcactctcaccatcaacagcctgcagcctgaagattttgcaacttactattgtcaacaggctaacagtttcccgctcactttcggcggagggaccaaggtggagatcaaacga

SEQ ID NO: 384 - protein, 108 aa, synthetic construct
  REGION 1..108: note = Synthetic
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 385 - DNA, 18 bp, synthetic construct
  misc_feature 1..18: note = Synthetic
  source 1..18: mol_type = other DNA
cagggtattagcaactgg

SEQ ID NO: 386 - protein, 6 aa, synthetic construct
  REGION 1..6: note = Synthetic
QGISNW

SEQ ID NO: 387-388 - skipped (2 entries)

SEQ ID NO: 389 - DNA, 27 bp, synthetic construct
  misc_feature 1..27: note = Synthetic
  source 1..27: mol_type = other DNA
caacaggctaacagtttcccgctcact

SEQ ID NO: 390 - protein, 9 aa, synthetic construct
  REGION 1..9: note = Synthetic
QQANSFPLT

SEQ ID NO: 391 - protein, 340 aa, synthetic construct
  REGION 1..340: note = Synthetic
EAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0390489 A1
(43) Pub. Date: Nov. 28, 2024

(54) METHODS OF INCREASING STRENGTH AND FUNCTIONALITY WITH GDF8 INHIBITORS

(71) Applicant: Regeneron Pharmaceuticals, Inc., Tarrytown, NY (US)

(72) Inventors: Robert C. PORDY, Ardsley, NY (US); Xiaobing QIAN, Briarcliff Manor, NY (US); Stephen DONAHUE, Princeton, NJ (US)

(21) Appl. No.: 18/674,399

(22) Filed: May 24, 2024

Related U.S. Application Data
(63) Continuation of application No. 17/149,540, filed on Jan. 14, 2021, now Pat. No. 12,029,788.
Application No. 17/149,540 is a continuation of application No. 15/099,701, filed on Apr. 15, 2016, now Pat. No. 10,934,349.
(60) Provisional application No. 62/147,853, filed on Apr. 15, 2015.
(60) Provisional application No. 62/234,899, filed on Sep. 30, 2015.
(60) Provisional application No. 62/261,528, filed on Dec. 1, 2015.

(51) Int. Cl. A61K 39/395; A61K 39/00; C07K 14/47; C07K 14/475; C07K 16/22

(52) U.S. Cl. CPC A61K 39/3955; A61K 2039/505; C07K 14/4703; C07K 14/4716; C07K 14/475; C07K 16/22; C07K 2317/21; C07K 2317/34; C07K 2317/56; C07K 2317/76

(57) ABSTRACT

The disclosure provides compositions, kits, and methods of using a GDF-8 inhibitor to increase lean muscle mass. In embodiments, a GDF-8 inhibiter is an antibody or antigen binding fragment thereof that specifically binds GDF-8. In embodiments, a method comprises providing an exercise regimen for the subject, and administering a composition comprising an effective amount of a GDF-8 inhibitor.

### CROSS-REFERENCE TO RELATED APPLICATIONS

This application is a Continuation application of U.S. patent application Ser. No. 15/099,701, filed Apr. 15, 2016, which claims the benefit of U.S. Provisional Application No. 62/147,853, filed Apr. 15, 2015, and U.S. Provisional Application No. 62/234,899, filed Sep. 30, 2015, and U.S. Provisional Application No. 62/261,528, filed Dec. 1, 2015. The disclosures of each which are incorporated herein in their entirety.

### REFERENCE TO A SEQUENCE LISTING

This application contains a Sequence Listing, which has been submitted electronically in XML format and is hereby incorporated by reference in its entirety. Said XML file, created on Aug. 7, 2024, in named Sequence-Listing-40848-0055USC2.xml and is 458,394 bytes in size.

### INTRODUCTION

A decrease in skeletal muscle mass appears to play a significant pathological role in the progression of a wide variety of disorders associated with aging, frailty, and certain metabolic conditions. In the elderly, conditions such as sarcopenia and specific events such as a hip fracture may be directly tied to a significant loss of global muscle mass. In both older and younger populations, the recovery from immobilization and orthopedic surgeries may be linked to the degree of acute muscle loss associated with both muscle disuse and atrophy driven by the procedure. In addition, the gain or maintenance of skeletal muscle mass can result in the prevention of obesity as well as metabolic improvements.

Myostatin or growth differentiation factor 8 (GDF8) is a soluble TGF-β superfamily ligand. It is a negative regulator of muscle growth expressed principally in skeletal muscle but low expression has been reported in other tissues such as heart and adipose, at levels approximately 100-fold lower than that seen in skeletal muscle (McPherron Nature 387:83, 1997, Sharma J. Cell. Phys. 180:1, 1999, Lee Annrev. Cell Dev. Biol. 20:61, 2004, Allen Pysiol. Rev. 88:257, 2008, Heineke Cir. 121:419, 2010). Mature myostatin is highly conserved among species, and inactivating mutations of the myostatin gene leads to a hypermuscular phenotype in multiple species including mice, cattle, dogs, and humans. Conversely, overexpression of myostatin in mice (by injection of transfected CHO cells into thighs of athymic mice or generation of striated muscle transgenic mice) caused a significant decrease in body mass due to decrease in muscle fiber size (McPherron 1997cied supra, Grobet Nat. Genet. 17:71, 1997, Mosher PLOS Genet. 3:e79, 2007, Schuelke NEJM 350:2682, 2004). While the myostatin null mouse phenotype demonstrates the importance of myostatin in the control of muscle size during development, hypertrophy can also be elicited in adult muscle through inhibition of myostatin by neutralizing antibodies, decoy receptors, or other antagonists. However, the effects on myostatin inhibitors on cardiac tissue in function may provide an undesirable side effect profile for use of these inhibitors to treat conditions like sarcopenia, and metabolic conditions.

### METHODS OF INCREASING STRENGTH AND FUNCTIONALITY WITH GDF8 INHIBITORS

This disclosure provides methods and formulations for use in the methods as described herein. GDF8 inhibitors are useful to enhance lean muscle mass in a subject, for example, in combination with exercise. In some embodiments, the subject is a subject that that does not have a disease or disorder that significantly limits the subject's ability to participate in resistance training. In embodiments, the disease or disorder is one in which a physician has recommended limited physical activity for the subject or in which exercise is contraindicated such as uncontrolled diabetes, recent myocardial infarction, unstable cardiac conditions, acute heart failure, severe myocarditis, uncontrolled hypertension, cardiac valve disease requiring surgery, and severe aortic stenosis.

In embodiments, a method for increasing lean body mass in a subject comprises providing an exercise regimen for the subject, and administering a composition comprising an effective amount of a GDF-8 inhibitor wherein, the effective amount is at least 400 mg. In embodiments, the exercise regimen includes, without limitation, resistance training, weight training, yoga, aerobic exercise, and pilates. In embodiments, a GDF8 inhibitor is an antibody or antigen binding fragment that specifically binds GDF8. In embodiments, the antibody or antigen binding fragment comprises heavy chain CDRs contained within a heavy chain variable region selected from the group consisting of SEQ ID NO: 2, SEQ ID NO: 18, SEQ ID NO: 34, SEQ ID NO: 50, SEQ ID NO: 66, SEQ ID NO: 82, SEQ ID NO: 98, SEQ ID NO: 114, SEQ ID NO: 130, SEQ ID NO: 146, SEQ ID NO: 162, SEQ ID NO: 178, SEQ ID NO: 194, SEQ ID NO: 210, SEQ ID NO: 226, SEQ ID NO: 242, SEQ ID NO: 258, SEQ ID NO: 274, SEQ ID NO: 290, SEQ ID NO: 306, SEQ ID NO: 360, and SEQ ID NO: 376. In embodiments, the method further comprises an antibody or antigen binding fragment that comprises light chain CDRS contained within a light chain variable regions selected from the group consisting of SEQ ID NOs: SEQ ID NO: 10, SEQ ID NO: 26, SEQ ID NO: 42, SEQ ID NO: 58, SEQ ID NO: 74, SEQ ID NO: 90, SEQ ID NO: 106, SEQ ID NO: 122, SEQ ID NO: 138, SEQ ID NO: 154, SEQ ID NO: 170, SEQ ID NO: 186, SEQ ID NO: 202, SEQ ID NO: 218, SEQ ID NO: 234, SEQ ID NO: 250, SEQ ID NO: 266, SEQ ID NO: 282, SEQ ID NO: 298, SEQ ID NO: 314, SEQ ID NO: 322, SEQ ID NO: 368, and SEQ ID NO: 384.

In embodiments, compositions are formulated to contain an effective amount of a GDF8 inhibitor to increase lean muscle mass. In embodiments, an effective amount is at least 0.1 mg/kg to about 10 gm/kg, 1 mg/kg to about 1 gm/kg, or 10 mg/kg to 100 mg/kg. In embodiments, the composition is administered at least once a week, twice a week, three times a week, four times a week, or five times a week. In embodiments, the exercise regimen is followed for at least 12 weeks. In embodiments, the compositions are formulated for intravenous, subcutaneous, or oral administration.

### BRIEF DESCRIPTION OF THE FIGURES

FIG. 1 (A) shows the results of the MAD study designed to assesses the safety, tolerability, pharmacokinetics (PK), immunogenicity, and pharmacodynamic (PD) effects of REGN1033 (anti-GDF8 antibody) administered subcutaneously (SC) in healthy volunteers 60 years of age and older. A total of 5 cohorts with 12 subjects enrolled in each cohort were studied. Subjects received SC doses of REGN1033 (n=9) or placebo (n=14). The planned REGN1033 dose regimens were 100, 200, or 400 mg Q2W for a total of 6 doses per subject and 200 mg or 400 mg Q4W for a total of 3 doses per subject. The % lean body mass was determined using dual energy x-ray absorptiometry (DEXA). FIG. 1 (B) shows the percent change in lean muscle mass per study visit per group in subjects receiving Placebo alone (third line from top), Placebo plus resistance training (RT) (bottom line), 400 mg SC of REGN1033 alone, for a total of 6 doses over the study period (second line from top), and 400 mg SC of REGN1033 plus RT (top line), for a total of 6 doses over the study period.

FIG. 2 shows the % change in appendicular fat mass per study visit for each group in subjects receiving Placebo alone (third line from top), Placebo plus resistance training (RT) (bottom line), 400 mg SC of REGN1033 alone (second line from top line), for a total of 6 doses over the study period, and 400 mg SC of REGN1033 plus RT (top line), for a total of 6 doses over the study period. Placebo alone and 400 mg SC of REGN1033 alone lines overlap one another.

FIG. 3 (A) shows percent change in thigh muscle mass including intramuscular fat per study visit per group in subjects receiving Placebo alone (third line from top), Placebo plus resistance training (RT) (bottom line), 400 mg SC of REGN1033 alone, for a total of 6 doses over the study period (second line from top), and 400 mg SC of REGN1033 plus RT (top line), for a total of 6 doses over the study period. FIG. 3 (B) shows percent change in thigh muscle mass excluding intramuscular fat per study visit per group in subjects receiving Placebo alone (third line from top), Placebo plus resistance training (RT) (bottom line), 400 mg SC of REGN1033 alone, for a total of 6 doses over the study period (second line from top), and 400 mg SC of REGN1033 plus RT (top line), for a total of 6 doses over the study period. Placebo alone and Placebo plus RT lines overlap one another.

FIG. 4 shows percent change in gynoid fat per study visit per group in subjects receiving Placebo alone (top line), Placebo plus resistance training (RT) (second line from top), 400 mg SC of REGN1033 alone, for a total of 6 doses over the study period (third line from top), and 400 mg SC of REGN1033 plus RT, for a total of 6 doses over the study period (bottom line). Placebo plus RT and 400 mg SC of REGN1033 alone lines overlap one another.

FIG. 5 shows percent change in chest press per study visit per group in subjects receiving Placebo alone (bottom line), Placebo plus resistance training (RT) (third line from top), 400 mg SC of REGN1033 alone, for a total of 6 doses over the study period (second line from top), and 400 mg SC of REGN1033 plus RT (top line), for a total of 6 doses over the study period.

### DETAILED DESCRIPTION

Before the present methods are described, it is to be understood that this disclosure is not limited to particular methods, and experimental conditions described, as such methods and conditions may vary. It is also to be understood that the terminology used herein is for the purpose of describing particular embodiments only, and is not intended to be limiting, since the scope of the invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the invention, the preferred methods and materials are now described. All patents, applications and non-patent publications mentioned in this specification are incorporated herein by reference in their entireties.

### DEFINITIONS

“Human Growth Differentiation Factor-8”, “GDF8” and “myostatin” are used interchangeably to refer to the protein encoded by the nucleic acid sequence of SEQ ID NO: 338 and the protein having the amino acid sequence of SEQ ID NO: 339 (propeptide) and SEQ ID NO: 340 (mature protein).

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-GDF8 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as diabodies, triabodies, tetrabodies and minibodies, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the invention include: (i) V_{H}-C_{H}1; (ii) V_{H}-C_{H}2; (iii) V_{H}-CH3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the invention using routine techniques available in the art.

The antibodies of the invention may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the invention in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al., Proc. Natl. Acad. Sci. (USA) 95:652-656 (1998)).

The term “specifically binds,” or the like, means that an antibody or antigen-binding fragment thereof forms a complex with an antigen that is relatively stable under physiologic conditions. Specific binding can be characterized by a dissociation constant of 1×10⁻⁶ M or less. Methods for determining whether two molecules specifically bind are well known in the art and include, for example, equilibrium dialysis, surface plasmon resonance, and the like. For example, an antibody that “specifically binds” human GDF8, as used in the context of the invention, includes antibodies that bind human GDF8 or portion thereof (e.g., a peptide comprising at least 6 contiguous amino acids of SEQ ID NO:340) with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. (See, e.g., Example 3, herein). An isolated antibody that specifically binds human GDF8 may, however, have cross-reactivity to other antigens, such as GDF8 molecules from other species.

The term “high affinity” antibody refers to those antibodies capable of binding to GDF8 with a dissociation constant (K_{D}) of about 10⁻⁸ M or less, about 10⁻⁹ M or less, about 10⁻¹⁰ M or less, about 10⁻¹¹ M or less, or about 10⁻¹² M or less, as measured by surface plasmon resonance, e.g., BIACORE™ or solution-affinity ELISA.

By the term “slow off rate” or “Koff” is meant an antibody that dissociates from GDF8 with a rate constant of 1×10⁻³ s⁻¹ or less, preferably 1×10⁻⁴ s⁻¹ or less, as determined by surface plasmon resonance, e.g., BIACORE™.

A “neutralizing” or “blocking” antibody, is intended to refer to an antibody whose binding to GDF8 results in inhibition of the biological activity of GDF8. This inhibition of the biological activity of GDF8 can be assessed by measuring one or more indicators of GDF8 biological activity. These indicators of GDF8 biological activity can be assessed by one or more of several standard in vitro or in vivo assays known in the art.

As used herein, the expression “anti-GDF8 antibody” also includes multispecific antigen-binding molecules (e.g., bispecific antibodies) wherein at least one binding domain (e.g., “binding arm”) of the multispecific antigen-binding molecule specifically binds GDF8.

Exemplary anti-GDF8 antibodies that can be used in the context of the invention include, e.g., the fully-human anti-GDF8 antibody H4H1657N2 (Regeneron/Sanofi) (e.g., an anti-GDF8 antibody comprising the heavy and light chain variable regions having amino acid sequences SEQ ID NO: 360 and SEQ ID NO: 368, respectively, as set forth in U.S. Pat. No. 8,840,894). Other GDF8 antagonists that can be used in the context of the methods of the invention include anti-GDF8 antibodies (e.g., the antibody designated 2_112_1 (ATCC deposit designation PTA-6574)) as set forth in U.S. Pat. No. 7,807,159, anti-GDF8 antibodies (e.g., 12A5-5) as set forth in U.S. Pat. No. 8,999,343 and US Publication No. 2013/0209489, anti-GDF8 antibodies (e.g., 10B3H8L5 and 10B3H8L5-Fc-disabled) as set forth in US Publication No. 2013/0142788, the anti-GDF8 antibody stamulumab/MYO-29 as set forth in, e.g., U.S. Pat. No. 8,940,874, anti-GDF8 antibodies (e.g., RK22/PF-0625616) as set forth in U.S. Pat. No. 8,415,459, anti-GDF8 antibodies (e.g., JA-16) as set forth in U.S. Pat. No. 7,731,961, anti-GDF8 antibodies (e.g., RK35) as set forth in U.S. Pat. No. 8,496,934, anti-GDF8 antibodies (e.g., OGD1.0.0) as set forth in U.S. Pat. No. 8,992,913, anti-GDF8 Fab molecules as set forth in European Patent No. 1 773 041 B1, anti-GDF8 antibodies (e.g., 41C1E4) as set forth in U.S. Pat. No. 7,632,499, and anti-GDF8 antibodies (e.g., C12, C12-N93H and/or 510C2) as set forth in, e.g., U.S. Pat. Nos. 7,635,760 and 8,063,188. The disclosures of all of the aforementioned patents and patent application publications are incorporated by reference herein in their entireties

The fully-human anti-GDF8 antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The invention includes antibodies, and antigen-binding fragments thereof, which are derived from any of the amino acid sequences disclosed herein, wherein one or more amino acids within one or more framework and/or CDR regions are back-mutated to the corresponding germline residue(s) or to a conservative amino acid substitution (natural or non-natural) of the corresponding germline residue(s) (such sequence changes are referred to herein as “germline back-mutations”). A person of ordinary skill in the art, starting with the heavy and light chain variable region sequences disclosed herein, can easily produce numerous antibodies and antigen-binding fragments which comprise one or more individual germline back-mutations or combinations thereof. In certain embodiments, all of the framework and/or CDR residues within the V_{H} and/or V_{L} domains are mutated back to the germline sequence. In other embodiments, only certain residues are mutated back to the germline sequence, e.g., only the mutated residues found within the first 8 amino acids of FRI or within the last 8 amino acids of FR4, or only the mutated residues found within CDR1, CDR2 or CDR3. Furthermore, the antibodies of the invention may contain any combination of two or more germline back-mutations within the framework and/or CDR regions, i.e., wherein certain individual residues are mutated back to the germline sequence while certain other residues that differ from the germline sequence are maintained. Once obtained, antibodies and antigen-binding fragments that contain one or more germline back-mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the invention.

The invention also includes anti-GDF8 antibodies comprising variants of any of the HCVR, LCVR, and/or CDR amino acid sequences disclosed herein having one or more conservative substitutions. For example, the invention includes anti-GDF8 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In one embodiment, the antibody comprises an HCVR having an amino acid sequence selected from SEQ ID NO:360 and 376 with 8 or fewer conservative amino acid substitutions. In another embodiment, the antibody comprises an HCVR having an amino acid sequence selected from SEQ ID NO:360 and 376 with 6 or fewer conservative amino acid substitutions. In another embodiment, the antibody comprises an HCVR having an amino acid sequence selected from SEQ ID NO:360 and 376 with 4 or fewer conservative amino acid substitutions. In another embodiment, the antibody comprises an HCVR having an amino acid sequence selected from SEQ ID NO:360 and 376 with 2 or fewer conservative amino acid substitutions. In one embodiment, the antibody comprises an LCVR having an amino acid sequence selected from SEQ ID NO:368 and 384 with 8 or fewer conservative amino acid substitutions. In another embodiment, the antibody comprises an LCVR having an amino acid sequence selected from SEQ ID NO:368 and 384 with 6 or fewer conservative amino acid substitutions. In another embodiment, the antibody comprises an LCVR having an amino acid sequence selected from SEQ ID NO:368 and 384 with 4 or fewer conservative amino acid substitutions. In another embodiment, the antibody comprises an LCVR having an amino acid sequence selected from SEQ ID NO:368 and 384 with 2 or fewer conservative amino acid substitutions.

In certain embodiments, antibody or antibody fragment of the invention may be conjugated to a therapeutic moiety (“immunoconjugate”), such as a cytotoxin, a chemotherapeutic drug, and immunosuppressant or a radioisotope.

An “isolated antibody,” as used herein, means an antibody that has been identified and separated and/or recovered from at least one component of its natural environment. For example, an antibody that has been separated or removed from at least one component of an organism, tissue or cell in which the antibody naturally exists or is naturally produced is an “isolated antibody” for purposes of the invention. An isolated antibody also includes an antibody in situ within a recombinant cell, as well as an antibody that has been subjected to at least one purification or isolation step. According to certain embodiments, an isolated antibody may be substantially free of other cellular material and/or chemicals.

The term “surface plasmon resonance”, as used herein, refers to an optical phenomenon that allows for the analysis of real-time biospecific interactions by detection of alterations in protein concentrations within a biosensor matrix, for example using the BIACORE™M system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. For example, an antibody is said to specifically bind an antigen when the K_{D} is less than or equal to 10⁻⁸ M, less than or equal to 10⁻⁹ M, or less than or equal to 10⁻¹⁰ M.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% w/w of a protein sample, usually about 95%, and preferably over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide analog or variant” as used herein refers to a polypeptide that is comprised of a segment of at least 25 amino acids that has substantial identity to a portion of an amino acid sequence and that has at least one of the following properties: (1) specific binding to GDF8 under suitable binding conditions, or (2) ability to block the biological activity of GDF8. Typically, polypeptide analogs or variants comprise a conservative amino acid substitution (or insertion or deletion) with respect to the naturally-occurring sequence. Analogs typically are at least 20 amino acids long, at least 50, 60, 70, 80, 90, 100, 150 or 200 amino acids long or longer, and can often be as long as a full-length naturally-occurring polypeptide.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton 1984 W. H. Freeman and Company, New York; Introduction to Protein Structure (Branden & Tooze, eds., 1991, Garland Publishing, NY); and Thornton et at. 1991 Nature 354:105, which are each incorporated herein by reference.

Non-peptide analogs are commonly used in the pharmaceutical industry as drugs with properties analogous to those of the template peptide. These types of non-peptide compound are termed “peptide mimetics” or “peptidomimetics” (see, for example, Fauchere (1986) J. Adv. Drug Res. 15:29; and Evans et al. (1987) J. Med. Chem. 30:1229, which are incorporated herein by reference. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo et al. (1992) Ann. Rev. Biochem. 61:387, incorporated herein by reference), for example, by adding internal cysteine residues capable of forming intramolecular disulfide bridges which cyclize the peptide.

As applied to polypeptides, the term “substantial identity” or “substantially identical” means that two peptide sequences, when optimally aligned, such as by the programs GAP or BESTFIT using default gap weights, share at least about 80% sequence identity, at least about 90%, at least about 95%, at least about 98% or at least about 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; and 6) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000), supra). Another preferred algorithm when comparing a sequence of the invention to a database containing a large number of sequences from different organisms is the computer program BLAST, especially blastp or tblastn, using default parameters. See, e.g., Altschul et al. (1990) J. Mol. Biol. 215:403-410 and Altschul et al. (1997) Nucleic Acids Res. 25:3389 402, each of which is herein incorporated by reference.

The length of polypeptide sequences compared for homology will generally be at least about 16 amino acid residues, at least about 20 residues, at least about 24 residues, at least about 28 residues, or at least about 35 residues. When searching a database containing sequences from a large number of different organisms, it is preferable to compare amino acid sequences.

The term “effective amount” is a concentration or amount of an antibody or antigen-binding fragment of an antibody which results in achieving a particular stated purpose. An “effective amount” of an anti-GDF8 antibody or antigen-binding fragment of an antibody thereof may be determined empirically. Furthermore, a “therapeutically effective amount” is a concentration or amount of an anti-GDF8 antibody or antigen-binding fragment thereof which is effective for achieving a stated therapeutic effect. This amount may also be determined empirically.

As used herein, a “healthy subject” refers to a subject that does not have a disease or disorder that significantly limits the subject's ability to participate in resistance training. In embodiments, the disease or disorder is one in which a physician has recommended limited physical activity for the subject or in which exercise is contraindicated such as uncontrolled diabetes, recent myocardial infarction, unstable cardiac conditions, acute heart failure, severe myocarditis, uncontrolled hypertension, cardiac valve disease requiring surgery, and severe aortic stenosis.

As used herein, “resistance training” refers to a set of exercises that causes a muscle to contract against an external resistance. The external resistance includes for example, a weight, a band, a kettleball, or the subject's body weight.

As used herein, “exercise regimen” refers to a plan of exercises. In embodiments, an exercise regimen includes exercises, such as, resistance training, weigh training, aerobic training, walking, interval training, yoga, and combinations thereof.

### Aspects of the Disclosure

The disclosure provides compositions, kits, and methods of using GDF-8 inhibitors to increase lean body mass. In embodiments, the GDF-8 inhibitor is an antibody or antigen binding fragment thereof that specifically binds GDF-8.

### Preparation of Human Antibodies

Methods for generating monoclonal antibodies, including fully human monoclonal antibodies are known in the art. Any such known methods can be used in the context of the invention to make human antibodies that specifically bind to GDF8.

Using VELOCIMMUNE™ technology or any other known method for generating monoclonal antibodies, high affinity chimeric antibodies to GDF8 are initially isolated having a human variable region and a mouse constant region. As in the experimental section below, the antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies of the instant invention possess very high affinities, typically possessing K_{D} of from about 10⁻¹² through about 10⁻⁹ M, when measured by binding to antigen either immobilized on solid phase or in solution phase. The mouse constant regions are replaced with desired human constant regions to generate the fully human antibodies of the invention, for example wild-type IgG1 (SEQ ID NO: 335) or IgG4 (SEQ ID NO: 336), or modified IgG1 or IgG4 (for example, SEQ ID NO: 337). While the constant region selected may vary according to specific use, high affinity antigen-binding and target specificity characteristics reside in the variable region.

### Antibodies or Antigen Binding Fragments Specific for GDF-8

The invention includes anti-GDF8 antibodies and antigen-binding fragments of antibodies which bind specific epitopes of human GDF8 (SEQ ID NO:340) and are capable of blocking the biological activity of GDF8. In one embodiment, the antibody or antigen-binding fragment thereof binds within an epitope comprising amino acids residues 1 to 109; 1 to 54; 1 to 44; 1 to 34; 1 to 24; and 1 to 14. In another embodiment, the antibody or antigen-binding fragment thereof binds within an epitope comprising of amino acid residues 65 to 72; 35 to 109; 45 to 109; 55 to 109; 65 to 109; 75 to 109; 85 to 109; 92 to 109; or 95 to 109. In another embodiment, the antibody or antigen-binding fragment thereof binds within an epitope comprising amino acid residue 48 to 72; 48 to 69; 48 to 65; 52 to 72; 52 to 65; or 56 to 65. In specific embodiments, the antibody or antigen-binding fragment thereof may bind within 2 or more epitopes.

The invention also includes antibodies and antigen-binding fragments thereof that bind wild-type mature GDF8 (SEQ ID NO: 340) but do not bind isolated peptides having less than the full amino acid sequence of SEQ ID NO: 340. For example, the invention includes anti-GDF8 antibodies that bind wild-type mature GDF8 (SEQ ID NO: 340) but do not bind isolated peptides consisting of 10 to 40 contiguous amino acids of SEQ ID NO: 340. The invention also includes anti-GDF8 antibodies that do not bind any linear epitopes within wild-type mature GDF8. In certain embodiments of the invention, the anti-GDF8 antibodies bind wild-type mature human GDF8 comprising SEQ ID NO: 340 but do not bind one or more isolated GDF8 peptides having an amino acid sequence selected from the group consisting of amino acids 1-14, 1-18, 17-42, 48-65, 48-69, 48-72, 52-65, 52-72, 56-65, 56-72, 65-72, 73-90, 75-105 and 91-105, of SEQ ID NO:340. In certain embodiments, the anti-GDF8 antibodies do not bind any of the aforementioned GDF8 peptides. Methods for determining whether a given antibody is able to bind a particular GDF8 peptide are known to persons of ordinary skill in the art.

The invention also includes isolated human antibodies, or antigen-binding fragments thereof, that specifically bind to wild-type mature human GDF8 (e.g., a protein or polypeptide comprising SEQ ID NO: 340), but do not bind to a chimeric GDF8 construct in which certain amino acids of GDF8 are replaced with the corresponding amino acid sequence(s) from a non-identical but related protein such as TGFβ-1. In one example, the chimeric construct is a GDF8/TGFβ-1 chimera in which amino acids 48-72 of mature GDF8 are replaced with the corresponding amino acid sequence of TGFβ-1 (e.g., amino acids 49-76 of TGFβ-1). An example of one such chimera is represented by SEQ ID NO: 352. Thus, in certain embodiments, the antibodies of the invention specifically bind to wild-type mature human GDF8 (SEQ ID NO: 340) but do not bind to the chimeric GDF8/TGFβ-1 construct of SEQ ID NO: 352, indicating that the epitope to which such antibodies bind includes or encompasses amino acids located within residues 48 to 72 of SEQ ID NO: 340. Blocking bioassays can also be used to indirectly ascertain if an antibody binds wild-type mature human GDF8 (SEQ ID NO: 340) and does not bind a chimeric GDF8/TGFβ-1 construct, e.g., the construct of SEQ ID NO: 352. For example, an antibody which blocks the bioactivity of wild-type mature human GDF8 but does not block the bioactivity of a chimeric GDF8/TGFβ-1 is deemed to bind to the portion of GDF8 that is replaced by the corresponding TGFβ-1 sequence in the chimeric construct.

Similarly, the invention also includes isolated human antibodies, or antigen-binding fragments thereof, that block wild-type mature GDF8-mediated activity in a bioassay but do not block activity of a chimeric GDF8 construct (e.g., a GDF8/TGFβ-1 chimera in which amino acids 48-72 of mature GDF8 are replaced with the corresponding amino acid sequence of TGFβ-1 (e.g., amino acids 49-76 of TGFβ-1).

The invention includes anti-GDF8 antibodies that bind to the same epitope as any of the specific exemplary antibodies described herein. Likewise, the invention also includes anti-GDF8 antibodies that cross-compete for binding to GDF8 or a GDF8 fragment with any of the specific exemplary antibodies described herein.

One can easily determine whether an antibody binds to the same epitope as, or competes for binding with, a reference anti-GDF8 antibody by using routine methods known in the art. For example, to determine if a test antibody binds to the same epitope as a reference anti-GDF8 antibody of the invention, the reference antibody is allowed to bind to a GDF8 protein or peptide under saturating conditions. Next, the ability of a test antibody to bind to the GDF8 molecule is assessed. If the test antibody is able to bind to GDF8 following saturation binding with the reference anti-GDF8 antibody, it can be concluded that the test antibody binds to a different epitope than the reference anti-GDF8 antibody. On the other hand, if the test antibody is not able to bind to the GDF8 molecule following saturation binding with the reference anti-GDF8 antibody, then the test antibody may bind to the same epitope as the epitope bound by the reference anti-GDF8 antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the invention, two antibodies bind to the same (or overlapping) epitope if, e.g., a 1-, 5-, 10-, 20-or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding with a reference anti-GDF8 antibody, the above-described binding methodology is performed in two orientations: In a first orientation, the reference antibody is allowed to bind to a GDF8 molecule under saturating conditions followed by assessment of binding of the test antibody to the GDF8 molecule. In a second orientation, the test antibody is allowed to bind to a GDF8 molecule under saturating conditions followed by assessment of binding of the reference antibody to the GDF8 molecule. If, in both orientations, only the first (saturating) antibody is capable of binding to the GDF8 molecule, then it is concluded that the test antibody and the reference antibody compete for binding to GDF8. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

The invention provides human or humanized antibodies and antigen-binding fragments of human or humanized antibodies that specifically bind human growth and differentiation factor 8 (GDF8). These antibodies are characterized by binding to GDF8 with high affinity and by the ability to neutralize GDF8 activity. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al. (2000) J. Immunol. 164:1925-1933).

In one embodiment, the antibody of the invention comprises a heavy chain variable region (HCVR) amino acid sequence selected from the group consisting of SEQ ID NO: 2, SEQ ID NO: 18, SEQ ID NO: 34, SEQ ID NO: 50, SEQ ID NO: 66, SEQ ID NO: 82, SEQ ID NO: 98, SEQ ID NO: 114, SEQ ID NO: 130, SEQ ID NO: 146, SEQ ID NO: 162, SEQ ID NO: 178, SEQ ID NO: 194, SEQ ID NO: 210, SEQ ID NO: 226, SEQ ID NO: 242, SEQ ID NO: 258, SEQ ID NO: 274, SEQ ID NO: 290, SEQ ID NO: 306, SEQ ID NO: 360, and SEQ ID NO: 376, or a substantially identical sequence thereof.

In one embodiment, the antibody of the invention comprises a light chain variable region (LCVR) amino acid sequence selected from the group consisting of SEQ ID NO: 10, SEQ ID NO: 26, SEQ ID NO: 42, SEQ ID NO: 58, SEQ ID NO: 74, SEQ ID NO: 90, SEQ ID NO: 106, SEQ ID NO: 122, SEQ ID NO: 138, SEQ ID NO: 154, SEQ ID NO: 170, SEQ ID NO: 186, SEQ ID NO: 202, SEQ ID NO: 218, SEQ ID NO: 234, SEQ ID NO: 250, SEQ ID NO: 266, SEQ ID NO: 282, SEQ ID NO: 298, SEQ ID NO: 314, SEQ ID NO: 322, SEQ ID NO: 368, and SEQ ID NO: 384 or a substantially identical sequence thereof.

In one embodiment, the antibody of the invention comprises a HCVR amino acid sequence and a LCVR amino acid sequence, wherein the HCVR/LCVR pair sequences are selected from the group consisting of SEQ ID NO: 2/10, SEQ ID NO: 18/26, SEQ ID NO: 34/42, SEQ ID NO: 50/58, SEQ ID NO: 66/74, SEQ ID NO: 82/90, SEQ ID NO: 98/106, SEQ ID NO: 114/122, SEQ ID NO: 130/138, SEQ ID NO: 146/154, SEQ ID NO: 162/170, SEQ ID NO: 178/186, SEQ ID NO: 194/202, SEQ ID NO: 210/218, SEQ ID NO: 226/234, SEQ ID NO: 242/250, SEQ ID NO: 258/266, SEQ ID NO: 274/282, SEQ ID NO: 290/298, SEQ ID NO: 306/314, SEQ ID NO: 114/322, SEQ ID NO: 360/368, and SEQ ID NO: 376/384.

The invention also features a human or humanized antibody or antigen-binding fragment of an antibody comprising a heavy chain complementarity determining region 3 (HCDR3) amino acid sequence and a light chain CDR3 amino acid sequence (LCDR3), wherein the HCDR3 amino acid sequence is selected from the group consisting of SEQ ID NO: 8, SEQ ID NO: 24, SEQ ID NO: 40, SEQ ID NO: 56, SEQ ID NO: 72, SEQ ID NO: 88, SEQ ID NO: 104, SEQ ID NO: 120, SEQ ID NO: 136, SEQ ID NO: 152, SEQ ID NO: 168, SEQ ID NO: 184, SEQ ID NO: 200, SEQ ID NO: 216, SEQ ID NO: 232, SEQ ID NO: 248, SEQ ID NO: 264, SEQ ID NO: 280, SEQ ID NO: 296, SEQ ID NO: 312, SEQ ID NO: 366, and SEQ ID NO: 382, or a substantially identical sequence thereof, and the LCDR3 amino acid sequence is selected from the group consisting of SEQ ID NO: 16, SEQ ID NO: 32, SEQ ID NO: 48, SEQ ID NO: 64, SEQ ID NO: 80, SEQ ID NO: 96, SEQ ID NO: 112, SEQ ID NO: 128, SEQ ID NO: 144, SEQ ID NO: 160, SEQ ID NO: 176, SEQ ID NO: 192, SEQ ID NO: 208, SEQ ID NO: 224, SEQ ID NO: 240, SEQ ID NO: 256, SEQ ID NO: 272, SEQ ID NO: 288, SEQ ID NO: 304, SEQ ID NO: 320, SEQ ID NO: 328, SEQ ID NO: 374, and SEQ ID NO: 390, or a substantially identical sequence thereof. In another embodiment, the antibody or fragment thereof comprises an HCDR3/LCDR3 amino acid sequence pair selected from the group consisting of SEQ ID NO: 8/16, SEQ ID NO: 24/32, SEQ ID NO: 40/48, SEQ ID NO: 56/64, SEQ ID NO: 72/80, SEQ ID NO: 88/96, SEQ ID NO: 104/112, SEQ ID NO: 120/128, SEQ ID NO: 136/144, SEQ ID NO: 152/160, SEQ ID NO: 168/176, SEQ ID NO: 184/192, SEQ ID NO: 200/208, SEQ ID NO: 216/224, SEQ ID NO: 232/240, SEQ ID NO: 248/256, SEQ ID NO: 264/272, SEQ ID NO: 280/288, SEQ ID NO: 296/304, SEQ ID NO: 312/320, SEQ ID NO: 120/328, SEQ ID NO: 366/374, and SEQ ID NO: 382/390.

In a related embodiment, the antibody or fragment thereof further comprises heavy chain CDR1 (HCDR1) and CDR2 (HCDR2) amino acid sequences and light chain CDR1 (LCDR1) and CDR2 (LCDR2) amino acid sequences, wherein the HCDR1 amino acid sequence is selected from the group consisting of SEQ ID NO: 4, SEQ ID NO: 20, SEQ ID NO: 36, SEQ ID NO: 52, SEQ ID NO: 68, SEQ ID NO: 84, SEQ ID NO: 100, SEQ ID NO: 116, SEQ ID NO: 132, SEQ ID NO: 148, SEQ ID NO: 164, SEQ ID NO: 180, SEQ ID NO: 196, SEQ ID NO: 212, SEQ ID NO: 228, SEQ ID NO: 244, SEQ ID NO: 260, SEQ ID NO: 276, SEQ ID NO: 292, SEQ ID NO: 308, SEQ ID NO: 362, and SEQ ID NO: 378, or a substantially identical sequence thereof; the HCDR2 amino acid sequence is selected from the group consisting of SEQ ID NO: 6, SEQ ID NO: 22, SEQ ID NO: 38, SEQ ID NO: 54, SEQ ID NO: 70, SEQ ID NO: 86, SEQ ID NO: 102, SEQ ID NO: 118, SEQ ID NO: 134, SEQ ID NO: 150, SEQ ID NO: 166, SEQ ID NO: 182, SEQ ID NO: 198, SEQ ID NO: 214, SEQ ID NO: 230, SEQ ID NO: 246, SEQ ID NO: 262, SEQ ID NO: 278, SEQ ID NO: 294, SEQ ID NO: 310, SEQ ID NO: 364, and SEQ ID NO: 380, or a substantially identical sequence thereof; the LCDR1 amino acid sequence is selected from the group consisting of SEQ ID NO: 12, SEQ ID NO: 28, SEQ ID NO: 44, SEQ ID NO: 60, SEQ ID NO: 76, SEQ ID NO: 92, SEQ ID NO: 108, SEQ ID NO: 124, SEQ ID NO: 140, SEQ ID NO: 156, SEQ ID NO: 172, SEQ ID NO: 188, SEQ ID NO: 204, SEQ ID NO: 220, SEQ ID NO: 236, SEQ ID NO: 252, SEQ ID NO: 268, SEQ ID NO: 284, SEQ ID NO: 300, SEQ ID NO: 316, SEQ ID NO: 324, SEQ ID NO: 370, and SEQ ID NO: 386 or a substantially identical sequence thereof; and the LCDR2 amino acid sequence is selected from the group consisting of SEQ ID NO: 14 (GVS), SEQ ID NO: 30 (AAS), SEQ ID NO: 46 (AAS), SEQ ID NO: 62 (AAS), SEQ ID NO: 78 (AAS), SEQ ID NO: 94 (AAS), SEQ ID NO: 110 (AAS), SEQ ID NO: 126 (RMS), SEQ ID NO: 142 (VAS), SEQ ID NO: 158 (AAS), SEQ ID NO: 174 (GAS), SEQ ID NO: 190 (ATS), SEQ ID NO: 206 (GAS), SEQ ID NO: 222 (GAS), SEQ ID NO: 238 (GAS), SEQ ID NO: 254 (GAS), SEQ ID NO: 270 (LGS), SEQ ID NO: 286 (GAS), SEQ ID NO: 302 (AAS), SEQ ID NO: 318 (LGS), SEQ ID NO: 326 (KIS), SEQ ID NO: 372 (TTS), and SEQ ID NO: 388 (AAS) or a substantially identical sequence thereof. In another embodiment, the HCDR1, HCDR2 and HCDR3 are selected from the group consisting of SEQ ID NO: 36/38/40, SEQ ID NO: 116/118/120, SEQ ID NO: 228/230/232, SEQ ID NO: 362/364/366, and SEQ ID NO: 378/380/382; and LCDR1, LCDR2 and LCDR3 are selected from the group consisting of SEQ ID NO: 44/46/48, SEQ ID NO: 124/126/128, SEQ ID NO: 236/238/240, SEQ ID NO: 370/372/374, and SEQ ID NO: 386/388/390. In yet another embodiment, the heavy and light chain CDRs are selected from the group consisting of SEQ ID NO: 36/38/40/44/46/48 (e.g. 21-E5), SEQ ID NO: 116/118/120/124/126/128 (e.g. 8D12), SEQ ID NO: 228/230/232/236/238/240 (e.g. 1A2), SEQ ID NO: 362/364/366/370/372/374 (e.g. H4H1657N2), and SEQ ID NO: 378/380/382/386/388/390 (e.g. H4H1669P).

In a related embodiment, the invention includes an antibody or antigen-binding fragment of an antibody which specifically binds GDF8, wherein the antibody or fragment comprises the heavy and light chain CDR domains contained within heavy and light chain variable domain sequences selected from the group consisting of SEQ ID NO: 2/10, SEQ ID NO: 18/26, SEQ ID NO: 34/42, SEQ ID NO: 50/58, SEQ ID NO: 66/74, SEQ ID NO: 82/90, SEQ ID NO: 98/106, SEQ ID NO: 114/122, SEQ ID NO: 130/138, SEQ ID NO: 146/154, SEQ ID NO: 162/170, SEQ ID NO: 178/186, SEQ ID NO: 194/202, SEQ ID NO: 210/218, SEQ ID NO: 226/234, SEQ ID NO: 242/250, SEQ ID NO: 258/266, SEQ ID NO: 274/282, SEQ ID NO: 290/298, SEQ ID NO: 306/314, SEQ ID NO: 114/322, SEQ ID NO: 360/368, and SEQ ID NO: 376/384.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The invention also provides nucleic acid molecules encoding the antibodies or antigen-binding fragments of the invention. Recombinant expression vectors carrying the antibody-encoding nucleic acids of the invention, and host cells into which such vectors have been introduced, are also encompassed by the invention, as are methods of making the antibodies of the invention by culturing the host cells of the invention.

In one embodiment, the antibody of the invention comprises CDRS contained within or a HCVR encoded by a nucleotide sequence selected from the group consisting of SEQ ID NO: 1, SEQ ID NO: 17, SEQ ID NO: 33, SEQ ID NO: 49, SEQ ID NO: 65, SEQ ID NO: 81, SEQ ID NO: 97, SEQ ID NO: 113, SEQ ID NO: 129, SEQ ID NO: 145, SEQ ID NO: 161, SEQ ID NO: 177, SEQ ID NO: 193, SEQ ID NO: 209, SEQ ID NO: 225, SEQ ID NO: 241, SEQ ID NO: 257, SEQ ID NO: 273, SEQ ID NO: 289, SEQ ID NO: 305, SEQ ID NO: 359, and SEQ ID NO: 375, or a substantially similar sequence having at least 95% homology thereof.

In one embodiment, the antibody of the invention comprises CDRS contained within or a LCVR encoded by a nucleotide sequence selected from the group consisting of SEQ ID NO: 9, SEQ ID NO: 25, SEQ ID NO: 41, SEQ ID NO: 57, SEQ ID NO: 73, SEQ ID NO: 89, SEQ ID NO: 105, SEQ ID NO: 121, SEQ ID NO: 137, SEQ ID NO: 153, SEQ ID NO: 169, SEQ ID NO: 185, SEQ ID NO: 201, SEQ ID NO: 217, SEQ ID NO: 233, SEQ ID NO: 249, SEQ ID NO: 265, SEQ ID NO: 281, SEQ ID NO: 297, SEQ ID NO: 313, SEQ ID NO: 321, SEQ ID NO: 367, and SEQ ID NO: 383 or a substantially similar sequence having at least 95% homology thereof.

The invention also features a fully human or humanized antibody or antibody fragment which binds GDF8 with an affinity (expressed as a dissociation constant, “K_{D}”) of about 1 nM or less, as measured by surface plasmon resonance assay (for example, BIACORE™). In certain embodiments, the antibody of the invention exhibits a K_{D} of about 700 pM or less; about 500 pM or less; about 320 pM or less; about 160 pM or less; about 100 pM or less; about 50 pM or less; about 10 pM or less; or about 5 pM or less.

In one embodiment, the invention provides a fully human or humanized monoclonal antibody (mAb) which specifically binds and inhibits human GDF8 and exhibits an IC₅₀ of less than or equal to about 10 nM; about 5 nM or less; about 3 nM or less; about 2 nM or less; about 1 nM or less; about 500 pM or less; or about 200 pM or less, as measured by GDF8 inducible luciferase assay. As shown in the experimental section below, some of the anti-GDF8 antibodies of the invention block the activity of closely related proteins, such as GDF11, with a much higher IC50 than GDF8 in a luciferase bioassay. In one embodiment, the invention provides an antibody or antigen-binding fragment of an antibody that exhibits at least about 10-fold, at least about 50-fold, at least about 100-fold, at least about 200-fold, at least about 500-fold, at least about 1000-fold, or at least about 1500-fold higher IC₅₀ for blocking GDF11 activity relative to GDF8.

The invention encompasses anti-GDF8 antibodies having a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of a galactosylation can be made in order to modify complement dependent cytotoxicity (CDC).

The invention includes anti-GDF8 antibodies which bind specific epitopes of GDF8 and are capable of blocking the biological activity of GDF8. In a first embodiment, the antibody of the invention binds an epitope of the mature GDF8 protein (SEQ ID NO:340) within amino acids from about 1 to about 109; from about 1 to about 54; from about 1 to about 44; from about 1 to about 34; from about 1 to about 24; and from about 1 to about 14. In a second embodiment, the antibody of the invention binds one or more of an epitope of the mature GDF8 protein (SEQ ID NO: 340) within amino acids from about 35 to about 109; from about 45 to about 109; from about 55 to about 109; from about 65 to about 109; from about 75 to about 109; from about 85 to about 109; from about 92 to about 109; or from about 95 to about 109. In a third embodiment, the antibody or antigen-binding fragment of the antibody binds within an epitope of the mature human GDF8 protein from about amino acid residue 48 to about 72; from about 48 to about 69; from about 48 to about 65; from about 52 to about 72; from about 52 to about 65; or from about 56 to about 65.

According to certain embodiments of the invention, the anti-GDF8 antibodies bind to human GDF8 but not to GDF8 from other species. Alternatively, the anti-GDF8 antibodies of the invention, in certain embodiments, bind to human GDF8 and to GDF8 from one or more non-human species. For example, the anti-GDF8 antibodies of the invention may bind to human GDF8 and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee GDF8.

The invention encompasses a human or humanized anti-GDF8 monoclonal antibody conjugated to a therapeutic moiety (“immunoconjugate”), such as a cytotoxin, a chemotherapeutic drug, an immunosuppressant or a radioisotope. Cytotoxin agents include any agent that is detrimental to cells. Examples of suitable cytotoxin agents and chemotherapeutic agents for forming immunoconjugates are known in the art, see for example, WO 05/103081, which is herein specifically incorporated by reference).

The antibodies of the invention may be monospecific, bi-specific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The anti-GDF8 antibodies of the invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the invention includes bi-specific antibodies wherein one arm of an immunoglobulin is specific for human GDF8 or a fragment thereof, and the other arm of the immunoglobulin is specific for a second therapeutic target or is conjugated to a therapeutic moiety.

An exemplary bi-specific antibody format that can be used in the context of the invention involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the invention.

The anti-GDF8 antibodies and antibody fragments of the invention encompass proteins having amino acid sequences that vary from those of the described antibodies, but that retain the ability to bind human GDF8. Such variant antibodies and antibody fragments comprise one or more additions, deletions, or substitutions of amino acids when compared to parent sequence, but exhibit biological activity that is essentially equivalent to that of the described antibodies. Likewise, the anti-GDF8 antibody-encoding DNA sequences of the invention encompass sequences that comprise one or more additions, deletions, or substitutions of nucleotides when compared to the disclosed sequence, but that encode an anti-GDF8 antibody or antibody fragment that is essentially bioequivalent to an anti-GDF8 antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. ioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-GDF8 antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-GDF8 antibody variants comprising amino acid changes which modify the glycosylation characteristics of the antibodies, e.g., mutations which eliminate or remove glycosylation.

### Therapeutic Administration and Formulations

The invention provides therapeutic compositions comprising the antibodies or antigen-binding fragments thereof of the invention. The administration of therapeutic compositions in accordance with the invention will be administered with suitable carriers, excipients, and other agents that are incorporated into formulations to provide improved transfer, delivery, tolerance, and the like.

A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the invention, provided that the active ingredient in the formulation is not inactivated by the formulation and the formulation is physiologically compatible and tolerable with the route of administration. See also Powell et al. “Compendium of excipients for parenteral formulations” PDA (1998) J Pharm Sci Technol 52:238-311.

The dose may vary depending upon the age and the size of a subject to be administered, target disease, conditions, route of administration, and the like. When the antibody of the invention is used for treating various conditions and diseases associated with GDF8, in an adult, it is advantageous to administer the antibody of the invention normally at a single dose of about 0.01 to about 20 mg/kg body weight, about 0.1 to about 10 mg/kg body weight, or about 0.1 to about 5 mg/kg body weight. Alternatively, the antibody or antigen binding fragment thereof can be administered to a healthy subject in combination with an exercise regimen. In embodiments, the healthy subject is experiencing age related loss of lean muscle mass and/or post-surgical muscle wasting. In embodiments, an effective amount for a subject is at least 400 mg, or about 36 mg/kg (assuming an average of 70 kg for an adult human).

Depending on the severity of the condition, the frequency and the duration of the treatment can be adjusted. In other parenteral administration and oral administration, the antibody can be administered in a dose corresponding to the dose given above. When the condition is especially severe, the dose may be increased according to the condition up to the amount that causes significant side effects, if any. In embodiments, the subject receives at least two doses or more subcutaneously. In embodiments, the subjects receive multiple doses intermittently, for example, once every week, once every two weeks, once every three weeks, once every four weeks, once every five weeks, and once every six weeks

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533).

A pharmaceutical composition of the invention can be delivered subcutaneously or intravenously with a standard needle and syringe. In addition, with respect to subcutaneous delivery, a pen delivery device readily has applications in delivering a pharmaceutical composition of the invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system, for example, with the use of a pump or polymeric materials. In another embodiment, a controlled release system can be placed in proximity of the composition's target, thus requiring only a fraction of the systemic dose.

Examples of the composition for oral administration include solid or liquid dosage forms, specifically, tablets (including dragees and film-coated tablets), pills, granules, powdery preparations, capsules (including soft capsules), syrup, emulsions, suspensions, etc. Such a composition is manufactured by publicly known methods and contains a vehicle, a diluent or an excipient conventionally used in the field of pharmaceutical preparations. Examples of the vehicle or excipient for tablets are lactose, starch, sucrose, magnesium stearate, and the like.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to 500 mg per dosage form in a unit dose; especially in the form of injection, it is preferred that the aforesaid antibody is contained in about 5 to 500 mg and in about 10 to 400 mg for the other dosage forms.

### Therapeutic Uses of the Antibodies

The antibodies of the invention are useful, inter alia, for increasing lean muscle mass in a subject. In some embodiments, a method of increasing lean muscle mass comprises providing an exercise regimen for the subject, and administering a composition comprising an effective amount of a GDF-8 inhibitor. In embodiments, the subject is a subject that has not been placed on exercise limitation by a physician and/or does not have a condition or disorder for which exercise is contraindicated. In embodiments, a subject has a loss of lean muscle mass. In other embodiments, the subject is at least 50 years, 55 years, or 60 years of age or older.

In embodiments, the antibodies of the invention are useful, inter alia, for the treatment, prevention and/or amelioration of any disease or disorder associated with GDF8 activity. More specifically, the antibodies of the invention are useful for the treatment of any condition or affliction which can be improved by increasing muscle strength/power and/or muscle mass and/or muscle function in an individual, or by favorably altering metabolism (carbohydrate, lipid and protein processing) by blocking GDF8 activity. Exemplary diseases, disorders and conditions that can be treated with the anti-GDF8 antibodies of the invention include, but are not limited to, sarcopenia, cachexia (either idiopathic or secondary to other conditions, e.g., cancer, chronic renal failure, or chronic obstructive pulmonary disease), muscle injury, muscle wasting and muscle atrophy, e.g., muscle atrophy or wasting caused by or associated with disuse, immobilization, bed rest, injury, medical treatment or surgical intervention (e.g., hip fracture, hip replacement, knee replacement, etc.) or by necessity of mechanical ventilation. Additional disorders that can be treated with the anti-GDF8 antibodies of the invention include, but are not limited to, sIBM (Sporadic Inclusion Body Myositis).

The invention includes therapeutic administration regimens which comprise administering an anti-GDF8 antibody of the invention in combination with at least one additional therapeutically active component. Non-limiting examples of such additional therapeutically active components include other GDF8 antagonists (e.g., small molecule inhibitors of GDF8 or other GDF8 antibodies or binding molecules), growth factor inhibitors, immunosuppressants, anti-inflammatory agents, metabolic inhibitors, enzyme inhibitors, and cytotoxic/cytostatic agents. The additional therapeutically active component(s) may be administered prior to, concurrent with, or after the administration of the anti-GDF8 antibody of the invention.

In a related embodiment, a method comprises combining administration of an antibody or antigen binding fragment thereof to a subject that is exercising on a regular basis, such as weight bearing exercises. In embodiments, a method comprises providing an exercise regimen, such as a regimen that promotes an increase in lean muscle mass. In embodiments, the exercise regimen includes one or more of resistance training, strength training, pilates, aerobic exercise, weight training, and yoga. In a specific embodiment, the exercise regimen includes resistance training and/or weight training. In embodiments, an exercise regimen is provided in writing and/or by illustration, on a computer readable medium, by video, or in an exercise facility.

In embodiments, resistance training includes training of any muscles of the body including without limitation, the muscles of a non-dominant hand, a dominant hand, a leg, an arm, a back, an abdominal muscle, a quadricep, a calf, a bicep, a tricep, a shoulder, a gluteus muscle, and/or a chest muscle. Resistance training can include the use of weights, a resistance band, exercise machines, and/or the subject's own body weight. Exercises can include chest press, leg press, arm curl, leg curl, hand grip, abdominal crunch, calf press, bicep curl, tricep curl, plank, side plank, and/or stair climb.

In embodiment, the exercise regimen provides for at least one set of exercises per muscle group and in specific embodiments, more than one, two or three sets of exercises. In embodiments, a set of exercises include at least eight repetitions or more.

A subject follows the provided exercise regimen at least once a week, twice a week, three times a week, four times a week, or five times a week. In embodiments, the exercise regimen is followed for at least 12 weeks.

In embodiments, a subject follows a set of exercises at a minimal level of intensity, for example, at 50% or less of the maximum weight that can be lifted for that exercise (1RM). In embodiments, the intensity is progressively increased during the exercise regimen. In embodiments, a 1-RM for each exercise (chest press, leg press, leg curl, and arm curl) can be measured at the exercise facility on the equipment used for exercise training at various time intervals such as baseline, week 4, and week 8. In embodiments, intensity is increased about 10% of the 1RM per week. In embodiments, intensity is at least 50%, and is maintained at about 65-90% of maximum.

In embodiments, a method further comprises monitoring the subjects resistance training comprising providing a device that measures training and/or providing a log book or computer program for tracking the repetitions, the duration, the intensity, and the frequency of the resistance training. In embodiments, a device includes an accelerometer, a dynamometer, a linear positioning device, and an actigraph.

In embodiments, a method comprises administering a GDF-8 inhibitor in an amount effective to increase lean muscle mass without resulting in adverse side effects on cardiac muscle and/or function. As discussed previously, GDF-8 inhibitors could be associated with cardiac hypertrophy. However, in embodiments, a dose and administration regimen is selected to minimize any effects on cardiac muscle and/or function. In embodiments, markers such as creatinine kinase, troponin, and the like are monitored during treatment. In embodiments, the treatment is discontinued if cardiac hypertrophy is observed, and/or if cardiac markers indicative of cardiac damage are elevated at least 20%.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Human GDF-8

Human anti-GDF8 antibodies were generated as described in U.S. Pat. No. 8,840,894. The exemplary GDF8 inhibitor used in the following Example is the human anti-GDF-8 antibody designated “H4H1657N2” (also referred to as REGN1033). H4H1657N2 has the following amino acid sequence characteristics: heavy chain variable region (HCVR) comprising SEQ ID NO:360; light chain variable domain (LCVR) comprising SEQ ID NO:368. The CDRs of REGN1033 comprise heavy chain complementarity determining region 1 (HCDR1) comprising SEQ ID NO:362; HCDR2 comprising SEQ ID NO:364; HCDR3 comprising SEQ ID NO:366; light chain complementarity determining region 1 (LCDR1) comprising SEQ ID NO:370; LCDR2 comprising SEQ ID NO:372; and LCDR3 comprising SEQ ID NO:374.

### Example 2: Clinical Trial of Safety and Bioeffect of Anti-GDF-8 With and Without Exercise

A randomized, double-blind, placebo-controlled, multicenter, parallel-group study of repeated doses of subcutaneous REGN1033 treatment effects on safety, body composition and muscle volume, muscle strength and stair climb function in 120 healthy male and female subjects with a sedentary lifestyle, who were 60 years of age and older was conducted. A 2×2 factorial design was used; up to 120 subjects (4 arms of 30 subjects each) were to be randomized in a 1:1:1:1 ratio to Placebo alone, REGN1033 (400 mg SC Q2W×6 doses) alone, Placebo+resistance exercise training (RT), or REGN1033+RT. Randomization was stratified by sex and by study site. The RT consisted of a center-based low intensity resistance exercise training at 50% of 1-repetition maximum (1-RM) twice a week for a total of 12 weeks with at least one progressive adjustment of exercise load during the study.

The primary objective of the study was to assess the effect of REGN1033, with and without exercise, on total lean body mass as measured by dual-energy x-ray absorptiometry (DEXA). Secondary objects include assessments of effects on safety and tolerability; effects on appendicular lean mass and fat mass by DEXA, effects on thigh muscle volume measured by MRI, upper and lower body strength by 1-repetition maximum methods, maximum hand grip strength, and stair climb power.

### Patient Selection

The target population for this study 120 healthy male and female subjects with a sedentary lifestyle, who were 60 years of age and older.

The inclusion criteria were as follows: 1. Men and women aged 60 years and older, with no significant health issues or conditions; 2. Sexually active males willing to use contraceptives and not to donate sperm during the study and through 4 months after the study; 3. Females require clinical confirmation of postmenopausal status (at least 12 months since last menses, confirmed by postmenopausal levels of FSH>20 mIU/ml, or surgically sterile); 4. Body mass index (BMI) between 19 and 35 kg/m2 inclusive; 5. With no condition that could limit participation in supervised resistance training exercise based on the PAR-Q; 6. Sedentary lifestyle defined by a score of ≤125 on the CHAMPS-18 physical activity questionnaire in the last 3 months; 7. Willing to maintain current diet and adhere to exercise programs described for the study and to not start any new dieting/weight management programs; 8. Willing and able to return for all clinic visits and complete all study-related procedures; 9. Provide complete study-related questionnaires; and 10. Provide the informed consent form (ICF).

The exclusion criteria for the study include those subjects that have been hospitalized or had major surgery, have osteoarthritis, rheumatologic diseases or orthopedic disorders which limit joint range of motion or ability to exercise; have gastrointestinal disorders, chronic kidney disease, cancer, pulmonary disease, cardiac disease, asthma, stroke with residual paresis, paralysis, multiple sclerosis, Parkinson Disease, cognitive impairment, psychiatric conditions that warrant acute or chronic therapeutic intervention (eg, major depressive disorder, bipolar disorder, panic disorder, schizophrenia), current or previous use of any drugs known to influence muscle mass or performance within 6 months, and unable to undergo MRI of the thighs

### Drug Administration

REGN1033 was supplied as a lyophilized drug product and was administered subcutaneously in this study. Each vial of lyophilized REGN1033 was reconstituted under aseptic conditions to a final concentration of 100 mg/mL. Placebo matching REGN1033 is prepared in the same formulation as REGN1033 without the addition of REGN1033. The volume for placebo was the same at each dose level. Subjects received 400 mg REGN1033 or placebo (combined with resistance training RT for subjects in groups 3 and 4) every 2 weeks for a total of 6 doses SC in the abdomen. The specific abdominal quadrant was documented.

### Study Design

This study had a screening period of 28 days (day −28 to day −1, screening/pretreatment), a drug treatment period (day 1 to day 71) and a 10 week follow up period after the last dose administration. Baseline measures for weight, strength measures and stair climb function, and echocardiography, were obtained during the screening period (day −14 to day −1). Baseline measures for DEXA and MRI were obtained between day −7±3 and day −1. For all other parameters, baseline measures were obtained on day 1 (baseline). The total duration of the study from first dose administration was approximately 20 weeks.

Subjects were screened from day −28 to day −1 and eligible subjects were randomized to 1 of the 4 groups on day 1: Placebo; Placebo and RT; REGN1033; REGN1033 and RT. Subject eligibility was determined by standard screening procedures as well as screening for cognitive impairment using the Mini-Mental State Examination (MMSE) questionnaire, and screening for a sedentary life-style using the Community Health Activities Model Program for Seniors (CHAMPS) 18 physical activity questionnaire.

Within 2 weeks prior to the start of dosing (day −14 to day −1) all subjects were familiarized with the exercises and muscle strength and function measurements: leg press, chest press, leg curl, arm curl, handgrip strength, and unloaded and loaded stair climb (8-steps). Baseline upper and lower body strength (as determined by 1-repetition maximum [1-RM] for chest press and leg press), maximum handgrip strength, and unloaded and loaded stair climb power were determined for all subjects within 2 weeks (day −14 to day −1) prior to the start of dosing. For each strength/function measure, 2 tests were conducted for subjects in all 4 treatment groups in 2 separate visits within 2 weeks before administration of the first dose of study drug to accommodate familiarization with and learning of the testing procedures. The average value of these 2 measures during these 2 testing sessions will be used as the baseline value.

The 1-RM for each RT exercise (chest press, leg press, leg curl, and arm curl) was measured at the exercise facility on the equipment was used for exercise training at baseline, week 4, and week 8 and which was used to calculate the load for RT training during weeks 1 to 4, 5 to 8, and 9 to 12. Exercise training was conducted twice weekly for the 12 week treatment period. At least 1 day separated each exercise session. Within 10 days prior to the start of dosing (day −7±3 days) all subjects underwent whole body DEXA to determine total and regional fat mass and appendicular lean mass. Subjects also underwent MRI of both thighs to determine muscle volume and SC and intramuscular fat. The values obtained served as the baseline for these parameters.

On day 1, and every 2 weeks thereafter for a total of 6 times, subjects received SC doses of study drug (400 mg of REGN1033 or placebo) administered in the abdomen as 2 injections of 2 ml each injection site. Subjects were observed for 30 minutes for vital signs and collection of adverse events (AEs), including occurrence of injection site reactions. Accelerometry was used to monitor subject's physical activity during the study.

Subjects in groups 3 and 4 returned to the site for supervised low-intensity resistance training of the major muscle groups of the upper and lower extremities using resistance training equipment twice a week for 12 weeks. All training exercises were performed at a relatively low intensity of 50% of the 1-RM for each exercise.

All subjects completing the study returned for laboratory and safety assessments at weeks 2, 4, 6, 8 and 10 on days 15, 29, 43, 57, and 71 (all with a visit window of ±3 days) when subsequent doses of study drug were administered. Subjects were followed for 10 weeks after the last dose administration (day 71) and returned to the clinic for laboratory and safety assessments at weeks 12, 14, 16, and 20 (days 85±3, 99±3, 113±3, and 141±3 [until the end of the last study visit]). Efficacy measures (DEXA, MRI, muscle strength and physical function) were obtained at screening and at week 12 (day 85±3). DEXA and muscle strength (double leg press, chest press, and maximum handgrip) and physical function (stair climb power) were determined at week 6 and week 20. Electrocardiograms were administered at day 1, week 4, week 8, week 12 and at the end of the study. Echocardiograms were conducted at week 12 (day 85±3 days).

All subjects were instructed to wear a GT3X Actigraph device (set at 30 Hz sampling rate) on the hip for the first 2 weeks on study from day 1 until the day 15 visit and another 14 day period between day 71 and day 85 (excluding the periods for center-resistance training exercise and during sleep). Subjects returned the device to the study site for downloading of data (day 15 and day 85). Time spent at various activity levels was determined based on activity threshold analysis using pre-defined cut-offs. Estimates of metabolic rates and energy expenditure were determined by predefined algorithms (Actigraph Corp).

### Sample Analysis and Statistics

The primary efficacy variable, i.e. the percent change in Total Lean Mass by dual energy x-ray absorptiometry (DEXA) from baseline to week 12, was analyzed using a mixed-effect model repeated measure (MMRM) approach. Secondary efficacy variables were analyzed in a similar fashion. The model included factors (fixed effects) for treatment (with 4 levels of R1033+RT, R1033 without RT, Placebo+RT and Placebo without RT), baseline stratum (gender), visit, baseline value and treatment-by-visit interaction as covariates. The comparison of the primary endpoint between REGN1033 without RT and Placebo without RT was made. Same comparison was made between REGN1033 with RT and Placebo with RT. The exercise effect was also explored.

### Results

Across all 4 treatment groups, 93.8-96.6% of randomized subjects completed their Week 12 visit. There was only one subject, in the Placebo alone treatment group, who discontinued the study due to an adverse event. The % of subjects in each of Placebo (PLC), Placebo+RT (PLC+RT), REGN1033 (R1033), and REGN1033+RT (R1033+RT) groups that received all 6 doses of study drug was: 87.5%, 93.1%, 84.4%, and 90.6%, respectively. There was no imbalance across groups in compliance with either treatment or RT. The demographics were balanced among the 4 groups except the Placebo group had a higher percentage (18.8%) of black subjects; and the Placebo+RT group had higher percentage (75.9%) and the REGN1033 group had lower percentage (56.3%) of subjects' age greater than 65 years. The baseline values for DEXA, MRI, muscle strength and physical function for each group did not significantly differ from one another. (data not shown)

The primary objective of the study was to assess the effect of REGN1033, with and without exercise, on total lean body mass as measured by dual-energy x-ray absorptiometry (DEXA). Secondary objects include assessments of effects on safety and tolerability; effects on appendicular lean mass and fat mass by DEXA, effects on thigh muscle volume measured by MRI, upper and lower body strength by 1-repetition maximum methods, maximum hand grip strength, and stair climb power. In addition, potential metabolic effects of REGN1033 on HbA1C and HOMA-IR were examined.

The primary efficacy variable, i.e. the percent change in Total Lean Mass by dual energy x-ray absorptiometry (DEXA) from baseline to week 12, was analyzed using a mixed-effect model repeated measure (MMRM) approach. The model included factors (fixed effects) for treatment (with 4 levels of R1033+RT, R1033 without RT, Placebo+RT and Placebo without RT), baseline stratum (gender), visit, baseline value and treatment-by-visit interaction as covariates. The comparison of the primary endpoint between REGN1033 without RT and Placebo without RT was made. Same comparison was made between REGN1033 with RT and Placebo with RT. The exercise effect was also explored. Secondary Efficacy Endpoints with continuous outcome were analyzed by a similar method for primary efficacy endpoint.

The doses and dose regimens for this study were selected based on the results from other studies.

In a completed SAD study, 76 healthy volunteers received REGN1033 at doses up to 10 mg/kg intravenously (IV) and 400 mg SC. Among these, an elderly cohort of 8 healthy volunteers 65 to 85 years old received 6 mg/kg IV of study drug. All doses were well tolerated; no clinically significant safety signals were observed. (data not shown)

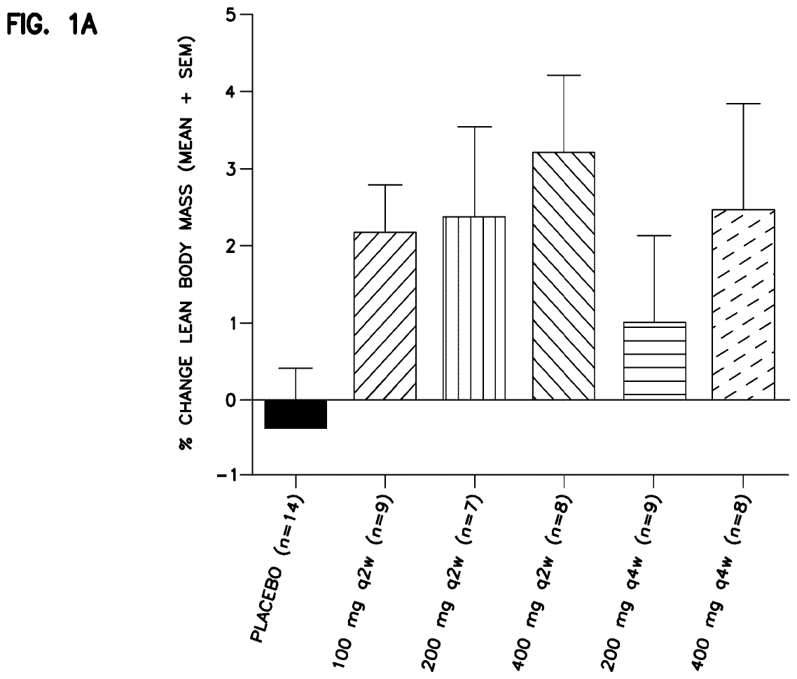

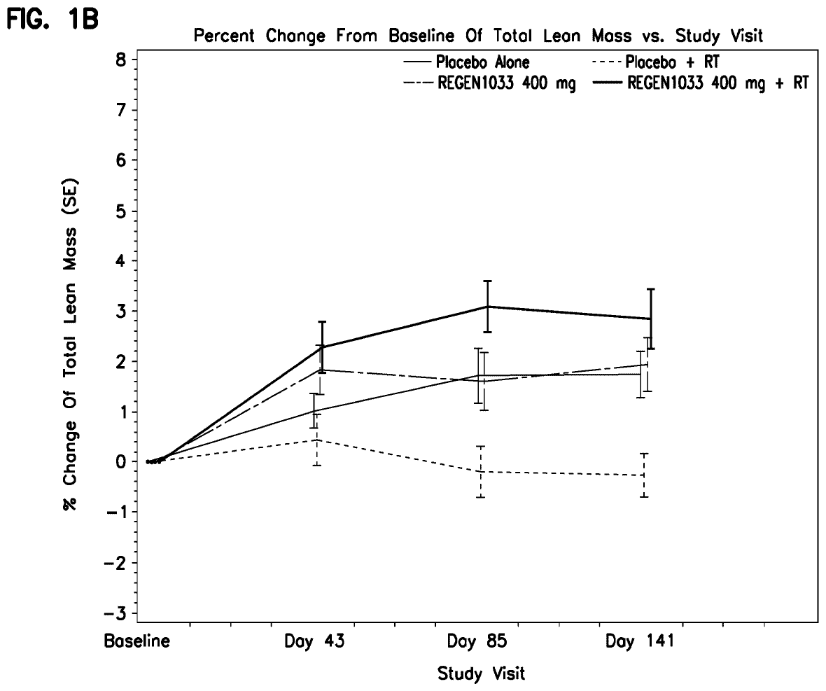

Another study was designed to assesses the safety, tolerability, pharmacokinetics (PK), immunogenicity, and pharmacodynamic (PD) effects of REGN1033 administered SC in healthy volunteers 60 years of age and older. A total of 5 cohorts with 12 subjects enrolled in each cohort were studied. Subjects received SC doses of REGN1033 (n=9) or placebo (n=3). The planned REGN1033 dose regimens were 100, 200, or 400 mg Q2W for a total of 6 doses per subject and 200 mg or 400 mg Q4W for a total of 3 doses per subject. The results from the study, shown in FIG. 1A, suggest that these doses of REGN1033 are well tolerated and are associated with an increase in lean mass detected by DEXA.

From these studies, PK and total GDF8 PD data suggest that REGN1033 may achieve concentrations associated with saturation of target engagement throughout the dose interval for the 400 mg Q4W and 400 mg Q2W dose levels. At the 100 mg Q4W dose level, there is expected to be REGN1033 exposures clearly less than those achieved at the higher doses. PK and PD data from phase 1 studies suggest that this lower dose will not achieve concentrations associated with engagement of the target across the entire dose interval in all individuals.

The results of the outcome of the primary efficacy variable are shown in Table 1 and FIG. 1B.

TABLE 1

| | | | | | | | | MMRM |
|---|---|---|---|---|---|---|---|---|
| | | | Week 12 | | | MMRM | MMRM | model |
| | Week 12 | Week 12 | REGN | Week 12 | Week 12 | model | model | REGN* |
| | REGN alone vs. | REGN + RT | combined vs. | PBO + RT | REGN + RT | REGN | Exercise | Exercise |
| | PBO alone | vs. PBO + RT | PBO combined | vs. PBO | VS. REGN | effect | Effect | Interaction |
| Summary of | | | | | | | | |
| Primary Efficacy | | | | | | | | |
| Percent change | 1.66% vs. 1.79% | 3.10% vs. −0.25% | 2.39% vs. 0.79% | −0.25% vs. 1.79% | 3.10% vs. 1.66% | (p = | (p = | (p = |
| in total lean | (p = 0.8640) | (p < 0.0001) | (p = 0.0048) | (p = 0.0088) | (p = 0.0609) | 0.0008) | 0.6333) | 0.0124) |
| mass by dual | | | | | | | | |
| energy x-ray | | | | | | | | |
| absorptiometry | | | | | | | | |
| (DXA) from | | | | | | | | |
| baseline | | | | | | | | |

Without exercise, REGN1033 (REGN) treatment did not increase total lean body mass measured by DEXA compared with Placebo (PBO) treatment (placebo adjusted change−0.13% p=0.8640, from baseline to week 12). With exercise, REGN1033 (REGN+RT) treatment significantly increased total lean body mass measured by DEXA from baseline to week 12 compared with Placebo (PBO+RT) treatment (placebo adjusted increase of 3.34%, p<0.0001). See Table 1 and FIG. 1B. The percent change in the ‘Placebo+RT’ group is significantly lower than in the ‘Placebo alone’ group: −0.25% vs. 1.79% (p=0.0088), but the ‘REGN1033+RT’ group is numerically higher than the ‘REGN1033 alone’ group: 3.10% vs. 1.66% (p=0.0609).

The factorial MMRM model also shows a significant REGN1033 treatment effect on percentage change of total lean body mass by DEXA from baseline to week 12 (P=0.0008) which is sustained at week 20 (P=0.0004). In addition, MMRM also shows an interaction effect (p=0.0124) between Exercise and Treatment.

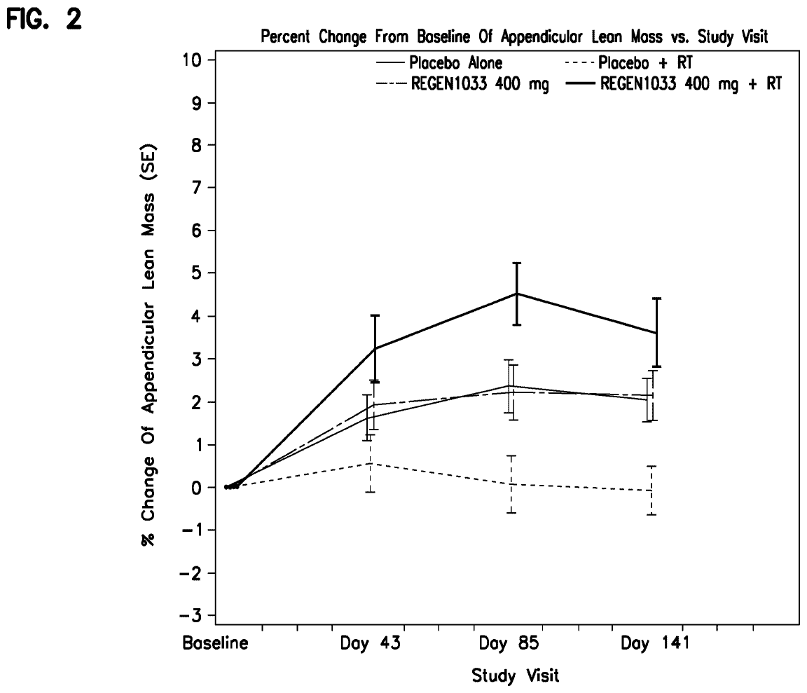

In subjects randomized to receive progressive resistance training exercise, REGN1033 (REGN+RT) treatment significantly increased the appendicular lean body mass measured by DEXA from baseline to week 12 compared with Placebo (PBO+RT) treatment (LS Mean difference vs Placebo 4.34%, P=0.001). As observed in the analysis of total lean body mass, the percent change in appendicular lean mass in the Placebo+RT group was significantly lower than in the Placebo alone group: 0.08% vs. 2.25% (p=0.0200), but the REGN1033+RT group was significantly higher than the REGN1033 alone group: 4.42% vs. 2.30% (p=0.0217). The factorial MMRM model also shows a significant treatment effect (0.0008) and interaction between treatment and exercise at week 12 (p=0.0040) which are sustained at week 20. See Table 2 and FIG. 2.

TABLE 2

| | | | | | | | | MMRM |
|---|---|---|---|---|---|---|---|---|
| | | | Week 12 | | | MMRM | MMRM | model |
| | Week 12 | Week 12 | REGN | Week 12 | Week 12 | model | model | REGN* |
| | REGN alone vs. | REGN + RT vs. | combined vs. | PBO + RT | REGN + RT | REGN | Exercise | Exercise |
| | PBO alone | PBO + RT | PBO combined | vs. PBO | vs. REGN | effect | Effect | Interaction |
| Percent | 2.30% vs. 2.25% | 4.42% vs. 0.08% | 3.37% vs. 1.19% | 0.08% vs. 2.25% | 4.42% vs. 2.30% | (p = | (p = | (p = |
| change in | d = 0.04% | d = 4.34% | d = 2.18% | (p = 0.0200) | (p = 0.0217) | 0.0008) | 0.9728) | 0.0040) |
| Appendicular | (p = 0.9617) | (p < 0.0001) | (p =0.0015). | | | | | |
| lean mass (g) | | | | | | | | |
| by DXA | | | | | | | | |

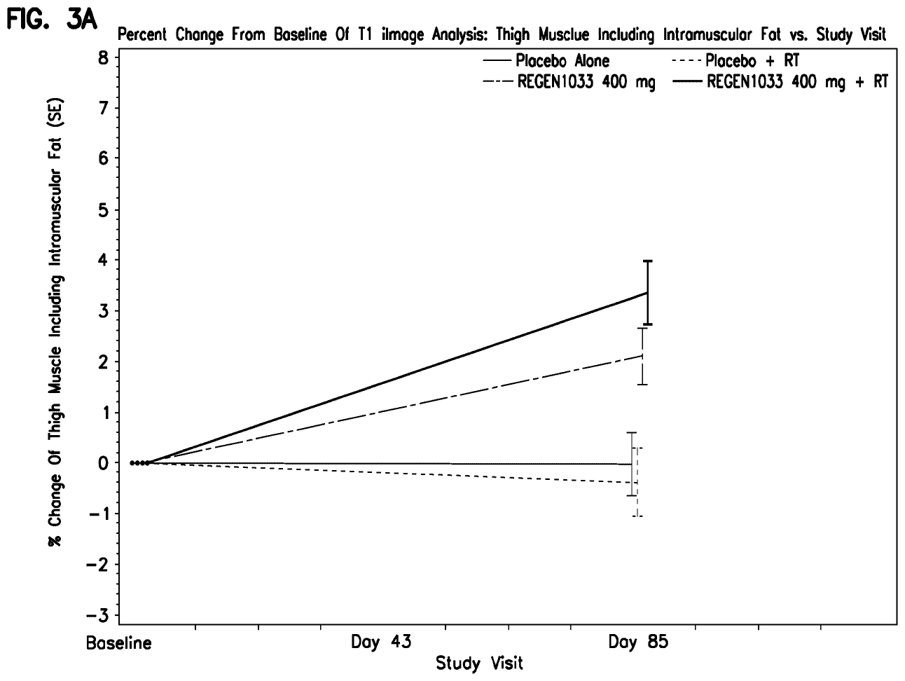

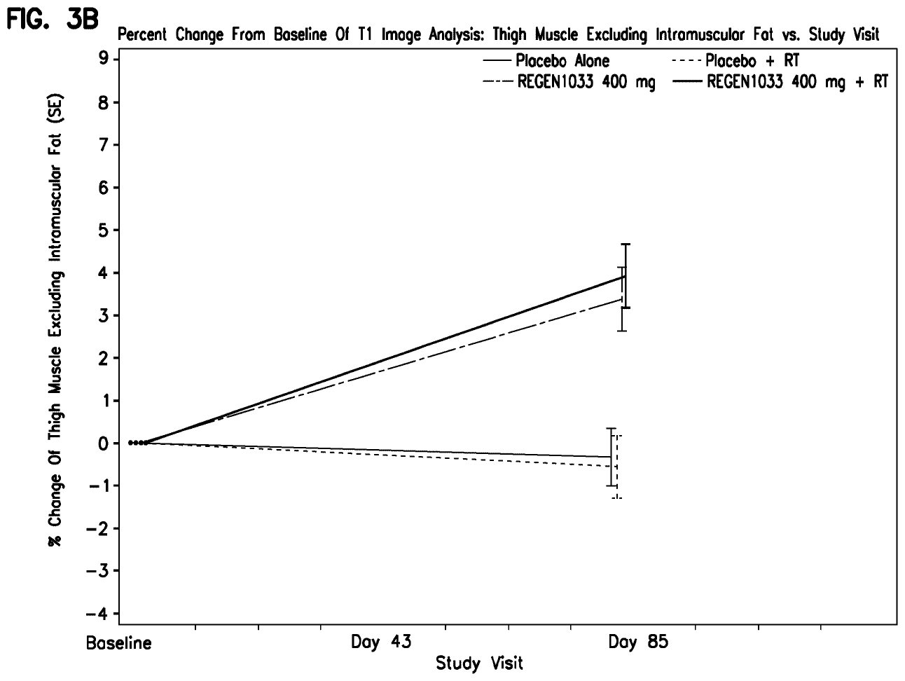

REGN1033 treatment, with or without exercise, resulted in statistically significant increases in MRI thigh muscle volume. See FIGS. 3A and 3B. The placebo adjusted effects on MRI thigh muscle volume (excluding intramuscular fat and vessels) at week 12 was +3.7%, p=0.0006 without exercise (REGN alone) and +4.5%, p<0.0001 with exercise (REGN+RT). There is a trend of increase of thigh muscle including intramuscular fat and vessels volume in the REGN1033 alone group compared to Placebo alone group and an increase of thigh muscle including intramuscular fat and vessels volume in the REGN1033+RT group as compared with Placebo+RT group. At week 12, the LS Mean percentage (%) change from baseline of thigh muscle including intramuscular fat and vessels volume (original scale) in Placebo alone, Placebo+RT, REGN1033 alone, and REGN1033+RT groups are −0.03%, −0.434%, 2.076%, and 3.405% respectively. In subjects randomized to not receive exercise, REGN1033 treatment significantly increased the thigh muscle including intramuscular fat and vessels volume measured by MRI from baseline to week 12 compared with Placebo treatment (LS Mean difference vs Placebo 2.11%%, P=0.0139). In subjects randomized to receive progressive resistance training exercise, REGN1033 treatment significantly increased the thigh muscle including intramuscular fat and vessels volume measured by MRI from baseline to week 12 compared with Placebo treatment (LS Mean difference vs Placebo 3.84%, P<0.0001).

The factorial MMRM model also shows a significant treatment effect (<0.0001) but not exercise effect (p=0.4453). The interaction between treatment and exercise at week 12 is not significant (p=0.1651). There was an increase of lean body mass by DEXA measures in the placebo group which was not accompanied by an increase of muscle volume by MRI measures. The reason for this discrepancy is unknown.

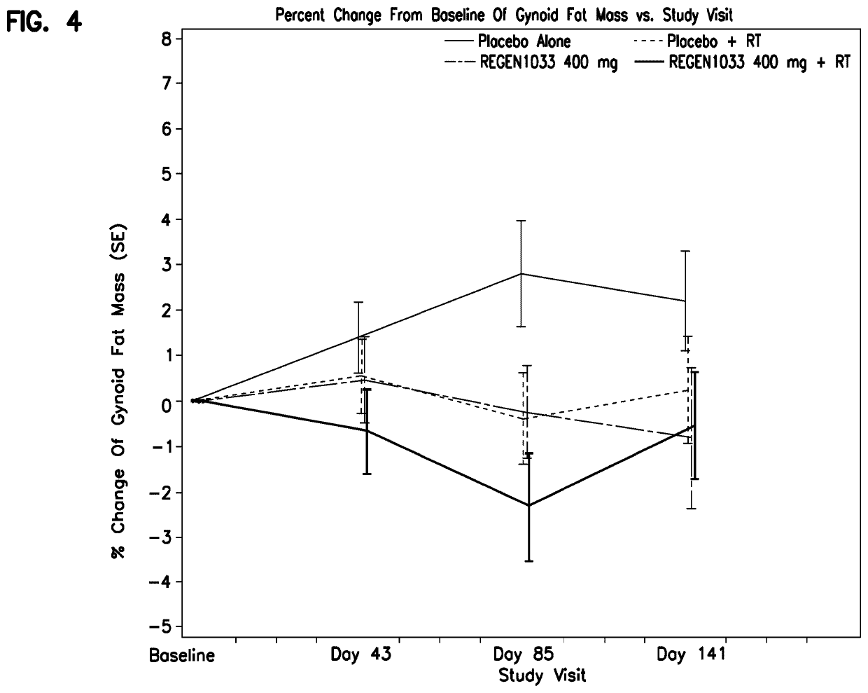

There is a trend of increase of Gynoid Fat Mass in the Placebo alone group at week 12 compared to other treatment groups and a decrease of Gynoid Fat Mass in the REGN1033+RT group. At week 12, the LS Mean percentage (%) change from baseline in Placebo alone, Placebo+RT, REGN1033 alone, and REGN1033+RT groups are 2.42%, −0.49%, −0.27%, and −2.17% respectively. See FIG. 4. The factorial MMRM model shows trend significant treatment (p=0.0824) and exercise (0.0531) effects on gynoid fat mass but no interaction between Exercise and Treatment (p=0.8774).

Data from four muscle strength measures were also obtained. Equipment was standardized across all study sites for the chest press and leg press 1-RM measures and not for arm curl and leg curl measures. Furthermore, only a subset of subjects had arm curl and leg curl measures.

There was a trend of increase of leg press strength in all groups. At week 12, the LS Mean percentage (%) change from baseline in Placebo alone, Placebo+RT, REGN1033 alone, and REGN1033+RT groups are 4.7%, 8.9%, 6.7%, and 9.7% respectively. The factorial MMRM model did not show significant treatment effect (p=0.8389) but a borderline exercise effect (p=0.0541) on leg press. The interaction effect between Exercise and Treatment is not significant (p=0.8029).

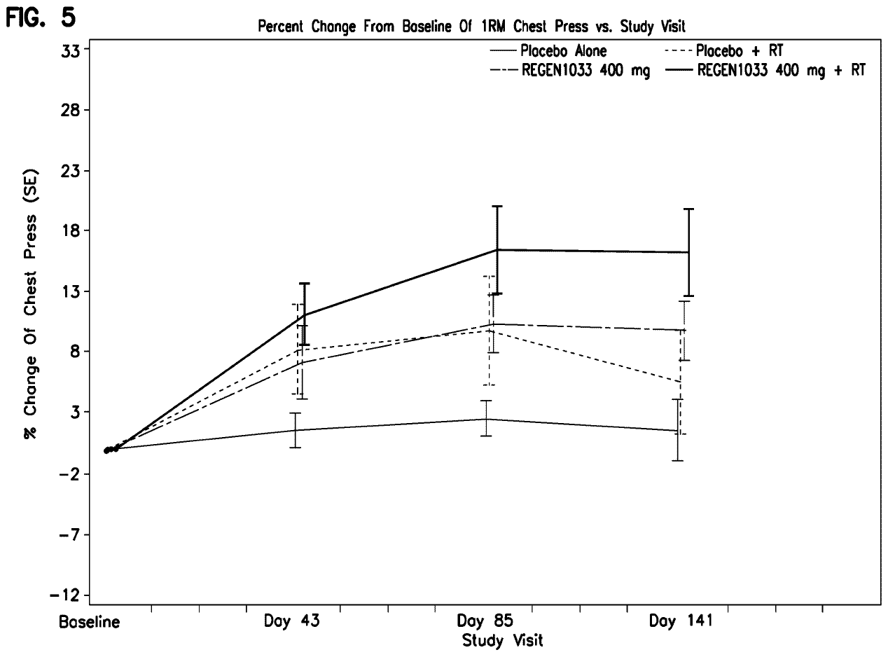

There was a trend of increase of chest press strength in all groups. The REGN1033 alone group increased more as compared to Placebo alone group, and the REGN1033+RT group also increased more as compared to Placebo+RT. See FIG. 5. At week 12, the LS Mean percentage (%) change from baseline in Placebo alone, Placebo+RT, REGN1033 alone, and REGN1033+RT groups are 2.2%, 10.5%, 10.4%, and 15.3% respectively. In subjects randomized to not receive progressive resistance training exercise, REGN1033 treatment increased chess press strength from baseline to week 12 compared with Placebo treatment (LS Mean difference vs Placebo 8.2%, P=0.0524). In subjects randomized to receive progressive resistance training exercise, REGN1033 treatment numerically increased chest press from baseline to week 12 compared with Placebo treatment, but not significant (LS Mean difference vs Placebo 5.3%, P=0.2316). The factorial MMRM model showed a significant treatment effects (p=0.0525), and a statistically significant exercise effect (0.0272) on chest press and no interaction effect (p=0.5089) between Exercise and Treatment.

There was a trend of increase of leg curl strength in all groups up to week 12. At week 12, the LS Mean percentage (%) change from baseline in Placebo alone, Placebo+RT, REGN1033 alone, and REGN1033+RT groups were 5.3%, 16.3%, 10.4%, and 9.7% respectively. The change in leg curl strength REGN1033 alone was 5.1% higher than Placebo alone, however, REGN1033+RT was 6.6% lower than Placebo+RT. The percent change in the Placebo+RT group was significant higher than in the ‘Placebo alone’ group: 16.3% vs. 5.30% (p=0.0050), however the REGN1033+RT group was not higher than the REGN1033 alone group: 9.79% vs. 10.4% (p=0.8388). The factorial MMRM model did not show a significant interaction effect (p=0.0229) between Exercise and Treatment.

There was a trend of increase of arm curl in all groups. (data not shown). At week 12, the LS Mean percentage (%) change from baseline in Placebo alone, Placebo+RT, REGN1033 alone, and REGN1033+RT groups are 8.1%, 21.7%, 12.4%, and 15.7% respectively. In subjects randomized to not receive progressive resistance training exercise, REGN1033 treatment increased arm curl strength from baseline to week 12 compared with Placebo, but this difference was not statistically significant (LS Mean difference vs Placebo 4.3%, P=0.4589).

In subjects randomized to receive progressive resistance training exercise, REGN1033 treatment increased less in arm curl strength from baseline to week 12 compared with Placebo, but not significant (LS Mean difference vs Placebo−6.0%, P=0.3121). The factorial MMRM model showed no significant REGN1033 treatment effect (p=0.8958), a borderline exercise (p=0.053) effects, and no significant interaction effect (p=0.1318) between Exercise and Treatment.

There was a trend of increase of the dominate hand grip strength in REGN1033 groups. (data not shown).At week 12, the LS Mean percentage (%) change from baseline in Placebo alone, Placebo+RT, REGN1033 alone, and REGN1033+RT groups were 1.8%, 1.2%, 4.8%, and 5.0% respectively. In subjects randomized to not receive progressive resistance training exercise, REGN1033 treatment increased the dominate hand grip strength from baseline to week 12 compared with Placebo, but not significant (LS Mean difference vs Placebo 3.0%, P=0.4180). In subjects randomized to receive progressive resistance training exercise, REGN1033 treatment increased more in the dominate hand grip strength from baseline to week 12 compared with Placebo, but not significant (LS Mean difference vs Placebo 3.8%, P=0.3198). The factorial MMRM model shows a statistically significant REGN1033 treatment effect (p=0.0407), but no exercise effect (p=0.7033) or interaction between Exercise and Treatment (p=0.8283).

There was a trend of increase of the non-dominate hand grip strength in REGN1033 groups but not in Placebo groups. (data not shown). At week 12, the LS Mean percentage (%) change from baseline in Placebo alone, Placebo+RT, REGN1033 alone, and REGN1033+RT groups were 1.8%, −0.8%, 7.0%, and 4.2% respectively. In subjects randomized to not receive progressive resistance training exercise, REGN1033 treatment increased more in the non-dominate hand grip strength from baseline to week 12 compared with Placebo, but not significant (LS Mean difference vs Placebo 5.1%, P=0.1432). In subjects randomized to receive progressive resistance training exercise, REGN1033 treatment increased more in the non-dominate hand grip strength from baseline to week 12 compared with Placebo, but not significant (LS Mean difference vs Placebo 5.0%, P=0.1708). The factorial MMRM model showed a statistically significant REGN1033 treatment effect (p=0.0039) and a borderline significance exercise effect (p=0.0581) on the non-dominate hand grip strength. There was no interaction effect between Exercise and Treatment (p=0.6224).

There was a trend of increase of loaded stair climb power in all groups. (data not shown) At week 12, the LS Mean percentage (%) change from baseline in Placebo alone, Placebo+RT, REGN1033 alone, and REGN1033+RT groups were 5.6%, 12.9%, 12.2%, and 15.0% respectively. In subjects randomized to not receive progressive resistance training exercise, REGN1033 treatment increased more in the loaded stair climb power from baseline to week 12 compared with Placebo, but not significant (LS Mean difference vs Placebo 6.6%, P=0.1303). In subjects randomized to receive progressive resistance training exercise, REGN1033 treatment increased more in loaded stair climb power from baseline to week 12 compared with Placebo, but not significant (LS Mean difference vs Placebo 2.2%, P=0.6368). The factorial MMRM model did not show a statistically significant treatment effect (p=0.3612) or interaction between Exercise and Treatment (p=0.8043), but showed a statistically significant exercise effect (p=0.0151) in loaded stair climb.

There was a trend of increase of unloaded stair climb power in all groups. At week 12, the LS Mean percentage (%) change from baseline in Placebo alone, Placebo+RT, REGN1033 alone, and REGN1033+RT groups were 6.1%, 12.5%, 5.4%, and 9.8% respectively. There was not difference between REGN1033 and the Placebo groups. The factorial MMRM model did not show a statistically significant treatment effect (p=0.4670) or interaction between Treatment and Exercise (p=0.4180) but showed a statistically significance Exercise effect (p=0.0295). The percent change in the Placebo+RT group was higher than in the Placebo alone group 12.5% vs. 6.1% (p=0.0900), and the REGN1033+RT group is higher than ‘REGN1033 alone’ group: 9.8% vs. 5.4% (p=0.2253), however these differences are not statistically significant.

REGN1033 treatment and the resistance training (RT) exercises were generally well tolerated in the study. The results for Treatment Adverse Events (TEAS) are shown in Table 3.

TABLE 3

| | Placebo | | REGN1033 | |
|---|---|---|---|---|
| | Alone | RT | 400 mg | 400 mg + RT |
| | (N = 32) | (N = 29) | (N = 32) | (N = 32) |
| Number of TEAEs | 151 | 103 | 129 | 98 |
| Number of serious | 1 | 1 | 3 | 0 |
| TEAEs | | | | |
| Subjects with at least | 26 | 27 | 27 | 28 |
| one TEAE | (81.3%) | (93.1%) | (84.4%) | (87.5%) |
| Subjects with at least | 10 | 6 | 14 | 10 |
| one drug related TEAE | (31.3%) | (20.7%) | (43.8%) | (31.3%) |
| Subjects with at least | 1 | 1 | 2 | 0 |
| one serious TEAE | (3.1%) | (3.4%) | (6.3%) | |
| Subjects with TEAEs | 3 | 1 | 1 | 0 |
| resulting in | (9.4%) | (3.4%) | (3.1%) | |
| discontinuation of | | | | |
| study drug | | | | |
| Subjects with a TEAE | 0 | 0 | 0 | 0 |
| resulting in death | | | | |

There were no deaths reported in this study and a total of 5 serious TEAS occurred in 4 subjects, including foot fracture in 1 subject in the Placebo Alone group; cholecystitis in 1 subject in the Placebo+RT group, stress cardiomyopathy in 1 subject in the REGN1033 group, and hypokalaemia and hypotension in 1 subject in the REGN1033 group. No serious TEAS were reported in the REGN1033+RT group. Subjects in Placebo Alone group reported more TEAEs and treatment-related TEAEs compared to other groups. The number and percentage of subjects experiencing at least one TEAE were similar across all treatment groups: 26 (81.3%) in placebo Alone, 27 (93.1%) in Placebo+RT, 27 (84.4%) in REGN1033, and 28 (87.5%) in REGN1033+RT group, respectively. A total of 5 subjects (3 and 1 in Placebo and Placebo+RT groups, compared to 1 and 0 subjects in REGN1033 and REGN1033+RT groups, respectively) discontinued drug treatment due to TEAEs.

Across the potentially clinically significant values (PCSV) categories of vital signs, ECG and hematology, there was no finding of an imbalance signaling higher frequency of PCSVs in the REGN1033 treatment groups. There were numerically more subjects with chemistry PCSVs in the REGN1033 Combined group than there were in the PLACEBO COMBINED group. Elevated Creatine Kinase values of >3×ULN occurred in 2 (6.3%). 1 (3.4%), 2 (6.3%) and 4 (12.5%) of subjects in the Placebo alone, Placebo+RT, REGN1033, and REGN1033+RT groups, respectively. There were no occurrence of Creatine Kinase values>10×ULN (Table 14.3.4.2.3). More subjects in the REGN1033 treated groups (7[10.9%]) had a body weight increase >5% than in the Placebo groups (3, [4.9%], Table 14.3.5.1.2).

A review of echocardiogram parameters related to cardiac structure and function did not reveal any signal of cardiac hypertrophy or any other deleterious effects on cardiac function. REGN1033 treatment had no effect on left ventricular ejection fraction, LV wall thickness or interventricular septum thickness. There were no reported increases in LV mass or LV mass index in either the REGN1033 or the Placebo treatment groups. (data not shown)

### Conclusions

With exercise, REGN1033 treatment significantly increased total lean body mass in healthy subjects measured by DEXA from baseline to week 12 compared with Placebo treatment (3.1% vs. −0.2%, <0.0001). Without exercise, REGN1033 treatment did not significantly increase total lean body mass measured by DEXA compared with Placebo treatment.

For secondary efficacy endpoints, the results of appendicular lean mass by DEXA are consistent with those observed in total lean body mass by DEXA. REGN1033 treatment, with or without exercise, resulted in statistically significant increases in MRI thigh muscle volume excluding intramuscular fat and vessels week 12 compared with Placebo treatment (placebo adjusted increase of 3.7%, P=0.0006 without exercise and 4.5%, P<0.0001 with exercise).

The resistance training (RT) exercises in this study were well tolerated in this elderly population. Significant exercise effects were shown by MMRM model in majority of the strength/function measures. This level of RT exercise did not result in significant increase of lean mass measured by DEXA or muscle volume measured by MRI. REGN1033 treatment also trended toward positive effects on several strength and functional endpoints examined in this study. Positive effects were seen in-chest press strength, handgrip strength, and loaded stair climb function.

Overall, REGN1033 SC 400 mg Q2W was generally well tolerated in this trial. The numbers of patients reporting TEAEs were comparable across treatment groups. A review of TEAEs did not reveal significant safety signal. Echocardiogram examinations did not reveal deleterious effects on cardiac structure or function.

### Example 3: Clinical Trial Protocol of Anti-GDF-8 Treatment of Subjects With Sarcopenia With and Without Exercise

A randomized, double-blind, placebo-controlled, multicenter phase 2 study of the safety and efficacy of 3-month SC REGN1033 treatment in patients with sarcopenia was conducted. Two hundred fifty patients were enrolled, in 4 treatment groups. Eligible patients were males and females with sarcopenia and associated mobility impairment, 70 years of age and older, with an average age of 78 years old. Patients were randomized in a 1:1:1:1 ratio to receive placebo SC every 2 weeks (Q2W) for a total of 6 treatments, REGN1033 at 300 mg SC Q2W for a total of 6 treatments, REGN1033 at 300 mg SC every 4 weeks (Q4W) for a total of 3 treatments (with placebo on alternating weeks), and REGN1033 at 100 mg SC Q4W for a total of 3 treatments (with placebo on alternating weeks). The study had a screening/pretreatment period (day −28 to day −1), a 12-week treatment period (day 1 to day 85), and an 8-week follow-up period (through day 141).

### Screening and Pretreatment Procedures (Day −28 to Day −1)

A sequential screening process took place across 3 visits, with initial eligibility determined at visit 1, and pretreatment procedures performed at visit 2 and visit 3. If feasible at the sites, visit 1 and visit 2 were conducted at the same time—if so, the visit 2 procedures were performed within 21 days of the first dose of study drug. Initial eligibility was determined at visit 1 by standard screening procedures, as well as 4-meter [4M] gait speed and the Mini-Mental State Examination (MMSE) score. Patients who met the initial eligibility criteria returned to the clinic at visit 2 and visit 3 for pretreatment baseline procedures and measurements.

The procedures included standard safety and laboratory assessments, DEXA scans, echocardiograms, strength measures (leg press, chest press, and handgrip strength), and function measures (stair climb, Short Physical Performance Battery [SPPB], 4M gait speed, and 6-Minute Walk Test [6MWT]).

### Treatment Period and Study Drug Administration (Day 1 to Day 85)

Starting on day 1, patients were randomized to receive either REGN1033 or matching placebo. The doses were as follows:
300 mg SC Q2W for a total of 6 treatments
300 mg SC Q4W for a total of 3 treatments (with placebo on alternating weeks to maintain the blind)
100 mg SC Q4W for a total of 3 treatments (with placebo on alternating weeks to maintain the blind)
Matching placebo SC Q2W for a total of 6 treatments

The injections were administered in the abdomen. Patients were observed for 30 minutes for vital signs and collection of adverse events (AEs), including occurrence of injection site reactions. Efficacy and safety procedures were performed, as well as patient-reported outcomes (PROs). Blood samples were collected for pharmacokinetics (PK), anti-drug antibodies (ADAs), and research. All blood samples were collected after an overnight fast and before dosing.

### Follow-Up (Day 86 to Day 141)

Follow-up visits were on day 141, 8 weeks after the end of treatment visit on day 85.

### Endpoints

The primary endpoint in the study was the percent change in total lean body mass measured by DEXA from baseline to week 12. The secondary endpoints were: TEAEs from baseline to the end of the study, changes from baseline in Appendicular lean mass by DEXA, maximal leg press strength, 1-repetition max (1-RM), maximal chest press strength (1-RM), 4M gait speed, SPPB and SPPB subscores, distance walked in the 6MWT, regional and total fat mass by DEXA, and hand grip strength by handheld dynamometer.

### Procedures and Assessments

Safety and tolerability of REGN1033 were assessed by vital signs, electrocardiogram (ECG), echocardiogram, Adverse events (AEs), and clinical laboratory evaluations. Patients were asked to monitor and report all AEs experienced from the time the informed consent is signed until the end of study visit.

Efficacy was assessed by DEXA, strength measures (leg press, chest press, and handgrip strength), and function measures (stair climb, SPPB, 4Mgait speed, and 6MWT). Other measures used were accelerometry and PROs (the 10-item Physical Function Form [PF-10], the Functional Assessment of Chronic Illness Therapy [FACIT] Fatigue Scale, the Health Assessment Questionnaire Disability Index [HAQ-DI], the Mini-Nutritional Assessment short form [MNA-SF]and the Rapid Assessment of Physical Activity [RAPA] questionnaire).

### Results

Ninety-five % of randomized patients completed the study. As shown below in Table 4, at each of the three dose regimens tested, REGN1033 treatment significantly increased total lean body mass from baseline to week 12 compared with placebo; mean differences from placebo were 1.7% (p=0.008), 1.8% (p=0.004) and 2.3% (p<0.001) for REGN1033 100 mg SC Q4W, 300 mg Q4W, and 300 mg Q2W respectively, corresponding to lean mass increases of 0.7, 0.8 and 1.0 kg. Appendicular lean mass also significantly increased in patients treated with REGN1033: placebo-adjusted changes ranged from 2.3-2.8%. DEXA-measured total fat mass, android fat mass, and gynoid fat mass all showed numerical decreases with REGN1033 treatment. REGN1033 treatment resulted in directionally greater mean changes from baseline in various measures of strength and function relative to placebo.

TABLE 4

| | | |
|---|---|---|
| REGN 100 mg Q4W | REGN 300 mg Q4W | REGN 300 mg Q2W |
| (n = 62) vs. PBO (n = 65) | (n = 64) vs. PBO (n = 65) | (n = 59) vs. PBO (n = 65) |

Primary Efficacy Endpoint: DEXA - Percent change from baseline to Week 12

| | | | |
|---|---|---|---|
| Percent change in total lean | 1.191% vs. −0.474% | 1.308% vs. −0.474% | 1.816% vs. −0.474% |
| mass by dual energy x-ray | d = 1.664% (p = 0.0077) | d = 1.781% (p = 0.0043) | d = 2.289% (p = 0.0004) |
| absorptiometry (DEXA) from | | | |
| baseline | | | |

Summary of Secondary Efficacy: DEXA - Percent change from baseline to Week 12

| | | | |
|---|---|---|---|
| Percent change in | 2.162% vs. −0.249% | 2.033% vs. −0.249% | 2.502% vs. −0.249% |
| Appendicular lean mass (g) | d = 2.412% (p = 0.0026) | d = 2.282% (p = 0.0043) | d = 2.751% (p = 0.0008) |
| by DEXA | | | |
| Percent change in Total fat | −0.077% vs. −0.076% | −2.666% vs. −0.076% | −0.947% vs. −0.076% |
| mass (g) determined by | d = −0.001% (p = 0.9993) | d = −2.590% (p = 0.0099) | d = −0.871% (p = 0.3945) |
| DEXA | | | |
| Percent change in Android | −0.496% vs. 2.501% | −3.164% vs. 2.501% | −2.209% vs. 2.501% |
| fat mass (g) by DEXA | d = −2.998% (p = 0.0587) | d = −5.665% (p = 0.0004) | d = −4.710% (p = 0.0038) |
| Percent change from | 0.022% vs. 0.156% | −2.608% vs. 0.156% | −2.185% vs. 0.156% |
| baseline in Gynoid fat | d = −0.135% (p = 0.9050) | d = −2.751% (p = 0.0152) | d = −2.341% (p = 0.0434) |
| mass(g) by DEXA | | | |

REGN1033 was generally safe and well tolerated. The frequency of adverse events was similar across treatment groups. The percentage of subjects experiencing at least one SAE was also similar across all treatment groups (7.7% in placebo group vs. 7.4% in REGN1033-treated groups). There was no discernable pattern to the distribution of SAEs. There were no clinically significant trends observed for laboratory tests, vital signs, ECGs and echocardiograms.

### Conclusions

REGN1033 treatment significantly increased total lean and appendicular lean mass in patients with sarcopenia and was well tolerated.

The invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. All publications referred to herein are hereby incorporated by reference.

TABLE 5

| SEQ ID NO: | Sequence | Mol_type | Synthetic |
|---|---|---|---|
| 13 | ggtgtatcc | DNA | Synthetic |
| 29 | gctgcatcc | DNA | Synthetic |
| 45 | gctgcatcc | DNA | Synthetic |
| 61 | gctgcatcc | DNA | Synthetic |
| 77 | gctgcatcc | DNA | Synthetic |
| 93 | gctgcatcc | DNA | Synthetic |
| 109 | gctgcatcc | DNA | Synthetic |
| 125 | cggatgtcc | DNA | Synthetic |
| 141 | gttgcatcc | DNA | Synthetic |
| 157 | gctgcatcc | DNA | Synthetic |
| 173 | ggtgcatcc | DNA | Synthetic |
| 189 | gctacatcc | DNA | Synthetic |
| 205 | ggtgcatcc | DNA | Synthetic |
| 221 | ggtgcatcc | DNA | Synthetic |
| 237 | ggtgcatcc | DNA | Synthetic |
| 253 | ggtgcatcc | DNA | Synthetic |
| 269 | ttgggttct | DNA | Synthetic |
| 285 | ggtgcatcc | DNA | Synthetic |
| 301 | gctgcatcc | DNA | Synthetic |
| 317 | ttgggttct | DNA | Synthetic |
| 325 | aagatttct | DNA | Synthetic |
| 329 | Xaa Xaa Xaa Xaa Xaa Xaa Xaa Xaa | PRT | Synthetic, VARIANT |
| | | | (1) . . . (1) Xaa = Gly |
| | | | (2) . . . (2) Xaa = Phe |
| | | | (3) . . . (3) Xaa = Thr |
| | | | (4) . . . (4) Xaa = Phe |
| | | | (5) . . . (5) Xaa = Ser |
| | | | (6) . . . (6) Xaa = Ala or Ser |
| | | | (7) . . . (7) Xaa = Phe or Tyr |
| | | | (8) . . . (8) Xaa = Gly or Ala |
| 330 | Xaa Xaa Xaa Xaa Xaa Xaa Xaa Xaa | PRT | Synthetic, VARIANT |
| | | | (1) . . . (1) Xaa = Ile |
| | | | (2) . . . (2) Xaa = Gly or Ser |
| | | | (3) . . . (3) Xaa = Tyr or Gly |
| | | | (4) . . . (4) Xaa = Ser or Asp |
| | | | (5) . . . (5) Xaa = Gly |
| | | | (6) . . . (6) Xaa = Gly |
| | | | (7) . . . (7) Xaa = Ser or Asn |
| | | | (8) . . . (8) Xaa = Ala or Glu |
| 331 | Xaa Xaa Xaa Xaa Xaa Xaa Xaa Xaa | PRT | Synthetic, VARIANT |
| | Xaa Xaa Xaa Xaa Xaa | | (1) . . . (1) Xaa = Ser or Ala |
| | | | (2) . . . (2) Xaa = Thr or Lys |
| | | | (3) . . . (3) Xaa = Asp or Ile |
| | | | (4) . . . (4) Xaa = Gly or Ser |
| | | | (5) . . . (5) Xaa = Ala or His |
| | | | (6) . . . (6) Xaa = Trp or Tyr |
| | | | (7) . . . (7) Xaa = Lys or Asp |
| | | | (8) . . . (8) Xaa = Met or Ile |
| | | | (9) . . . (9) Xaa = Ser or Leu |
| | | | (10) . . . (10) Xaa = Gly or Ser |
| | | | (11) . . . (11)Xaa = Leu or Gly |
| | | | (12) . . . (12)Xaa = Asp or Met |
| | | | (13) . . . (13)Xaa = Val or Asp |
| | | | (14) . . . (14)Xaa = Val or absent |
| 332 | Xaa Xaa Xaa Xaa Xaa Xaa | PRT | Synthetic, VARIANT |
| | | | (1) . . . (1) Xaa = Gln |
| | | | (2) . . . (2) Xaa = Asp or Gly |
| | | | (3) . . . (3) Xaa = Ile |
| | | | (4) . . . (4) Xaa = Ser |
| | | | (5) . . . (5) Xaa = Asp or Asn |
| | | | (6) . . . (6) Xaa = Tyr or Trp |
| 333 | Xaa Xaa Xaa | PRT | Synthetic, VARIANT |
| | | | (1) . . . (1) Xaa = Thr or Ala |
| | | | (2) . . . (2) Xaa = Thr or Ala |
| | | | (3) . . . (3) Xaa = Ser |
| 334 | Xaa Xaa Xaa Xaa Xaa Xaa Xaa Xaa | PRT | Synthetic, VARIANT |
| | Xaa | | (1) . . . (1) Xaa = Gln |
| | | | (2) . . . (2) Xaa = Lys or Gln |
| | | | (3) . . . (3) Xaa = Ala or Tyr |
| | | | (4) . . . (4) Xaa = Asp or Asn |
| | | | (5) . . . (5) Xaa = Ser |
| | | | (6) . . . (6) Xaa = Ala or Phe |
| | | | (7) . . . (7) Xaa = Pro |
| | | | (8) . . . (8) Xaa = Leu |
| | | | (9) . . . (9) Xaa = Thr |
| 371 | actacatcc | DNA | Synthetic |
| 387 | gctgcatcc | DNA | Synthetic |

1. A method for increasing lean body mass in a subject comprising:
a. providing an exercise regimen for the subject; and
b. administering a composition comprising an effective amount of a Growth and Differentiation Factor-8 (GDF-8) inhibitor.

2. The method of claim 1, wherein the effective amount is at least 400 mg.

3. The method of claim 1, wherein the effective amount comprises a dosing regimen selected from a group consisting of at least about 0.01 to about 20 mg/kg, about 0.1 mg/kg to about 10 mg/kg, about 0.1 to about 5 mg/kg body weight, about 1 mg/kg to about 10 mg/kg, and about 10 mg/kg to about 100 mg/kg.

4. (canceled)

5. The method of claim 1, wherein the exercise regimen comprises a physical activity selected from the group consisting of resistance training, yoga, aerobic exercise, walking, interval training, and pilates.

6. The method of claim 5, wherein the resistance training includes a set of exercises for training of a muscle selected from the group consisting of the dominant hand, non-dominant hand, leg, arm, chest and combinations thereof.

7. The method of claim 1, wherein the composition is administered at least once every six weeks, once every five weeks, once every four weeks, once every three weeks, once every two weeks, once a week, twice a week, three times a week, four times a week, or five times a week.

8. The method of claim 1, wherein the composition is formulated for intravenous, subcutaneous, or oral administration.

9. The method of claim 1, wherein the composition is formulated for subcutaneous delivery.

10. The method of claim 1, wherein the GDF-8 inhibitor is an antibody or antigen binding fragment that specifically binds GDF-8.

11. The method of claim 10, wherein the antibody or antigen binding fragment thereof comprises
three heavy chain complementarity determining regions (HCDRs) (HCDR1, HCDR2, HCDR3) contained within a heavy chain variable region (HCVR) amino acid sequence selected from the group consisting of SEQ ID NO: 2, SEQ ID NO: 18, SEQ ID NO: 34, SEQ ID NO: 50, SEQ ID NO: 66, SEQ ID NO: 82, SEQ ID NO: 98, SEQ ID NO: 114, SEQ ID NO: 130, SEQ ID NO: 146, SEQ ID NO: 162, SEQ ID NO: 178, SEQ ID NO: 194, SEQ ID NO: 210, SEQ ID NO: 226, SEQ ID NO: 242, SEQ ID NO: 258, SEQ ID NO: 274, SEQ ID NO: 290, SEQ ID NO: 306, SEQ ID NO: 360, and SEQ ID NO: 376; and
three light chain complementarity determining regions (LCDRs) (LCDR1, LCDR2, LCDR3) contained within a light chain variable region (LCVR) amino acid sequence selected from the group consisting of SEQ ID NO:10, SEQ ID NO:26, SEQ ID NO:42, SEQ ID NO:58, SEQ ID NO:74, SEQ ID NO:90, SEQ ID NO: 106, SEQ ID NO: 122, SEQ ID NO: 138, SEQ ID NO: 154, SEQ ID NO: 170, SEQ ID NO: 186, SEQ ID NO:202, SEQ ID NO:218, SEQ ID NO: 234, SEQ ID NO:250, SEQ ID NO:266, SEQ ID NO:282, SEQ ID NO:298, SEQ ID NO: 314, SEQ ID NO:322, SEQ ID NO:368, and SEQ ID NO:384.

12. (canceled)

13. The method of claim 10, wherein the antibody or antigen binding fragment thereof comprises three HCDRs (HCDR1, HCDR2, HCDR3) within a HCVR amino acid sequence and three LCDRs (LCDR1, LCDR2, LCDR3) within a LCVR amino acid sequence, wherein the HCVR/LCVR sequence pair is selected from the group consisting of SEQ ID NO: 2/10, SEQ ID NO: 18/26, SEQ ID NO: 34/42, SEQ ID NO: 50/58, SEQ ID NO: 66/74, SEQ ID NO: 82/90, SEQ ID NO: 98/106, SEQ ID NO: 114/122, SEQ ID NO: 130/138, SEQ ID NO: 146/154, SEQ ID NO: 162/170, SEQ ID NO: 178/186, SEQ ID NO: 194/202, SEQ ID NO: 210/218, SEQ ID NO: 226/234, SEQ ID NO: 242/250, SEQ ID NO: 258/266, SEQ ID NO: 274/282, SEQ ID NO: 290/298, SEQ ID NO: 306/314, SEQ ID NO: 114/322, SEQ ID NO: 360/368, and SEQ ID NO: 376/384.

14.-24 (canceled)

25. The method of claim 10, wherein the antibody or antigen binding fragment thereof comprises
the HCDR1, HCDR2 and HCDR3 amino acid sequences selected from the group consisting of SEQ ID NO: 36/38/40, SEQ ID NO: 116/118/120, SEQ ID NO: 228/230/232, SEQ ID NO: 362/364/366, and SEQ ID NO: 378/380/382; and
the LCDR1, LCDR2 and LCDR3 amino acid sequences selected from the group consisting of SEQ ID NO: 44/46/48, SEQ ID NO: 124/126/128, SEQ ID NO: 236/238/240, SEQ ID NO: 370/372/374, and SEQ ID NO: 386/388/390.

26. (canceled)

27. The method of claim 25, wherein the HCVR1, HCVR2, HCVR3, LCDR1, LCDR2, and LCDR3 amino acid sequences are selected from the group consisting of SEQ ID NO: 36/38/40/44/46/48, SEQ ID NO: 116/118/120/124/126/128, SEQ ID NO: 228/230/232/236/238/240, SEQ ID NO: 362/364/366/370/372/374, and SEQ ID NO: 378/380/382/386/388/390, respectively.

28. The method of claim 10, wherein the antibody or antigen binding fragment thereof comprises the heavy and light chain variable domain regions (HCVR/LCVR) comprising the amino acid sequence pairs selected from the group consisting of SEQ ID NO: 2/10, SEQ ID NO: 18/26, SEQ ID NO: 34/42, SEQ ID NO: 50/58, SEQ ID NO: 66/74, SEQ ID NO: 82/90, SEQ ID NO: 98/106, SEQ ID NO: 114/122, SEQ ID NO: 130/138, SEQ ID NO: 146/154, SEQ ID NO: 162/170, SEQ ID NO: 178/186, SEQ ID NO: 194/202, SEQ ID NO: 210/218, SEQ ID NO: 226/234, SEQ ID NO: 242/250, SEQ ID NO: 258/266, SEQ ID NO: 274/282, SEQ ID NO: 290/298, SEQ ID NO: 306/314, SEQ ID NO: 114/322, SEQ ID NO: 360/368, and SEQ ID NO: 376/384.

29.-84 (canceled)

85. The method of claim 1, wherein the subject is experiencing age related loss of lean muscle mass and/or post-surgical muscle wasting.

86. The method of claim 1, wherein the subject is 60 years of age or older.

87. The method of claim 1, wherein the subject is a human subject.

88.-92. (canceled)

93. The method of claim 1, wherein the subject is experiencing or at risk of age-related loss of lean muscle mass and/or a disease, disorder, or condition associated with a decrease in skeletal muscle.

94. The method of claim 93, wherein the disease, disorder, or condition is selected from the group consisting of sarcopenia, cachexia, muscle injury, muscle wasting, muscle atrophy, metabolic condition, and sporadic inclusion body myositis (sIBM).

95. The method of claim 94, wherein the disease, disorder, or condition is sarcopenia.

96. The method of claim 5, wherein the resistance training comprises weight training, use of resistance band, use of exercise machine, and/or use of the subject's own body weight.

97. The method of claim 1, wherein the increase in lean mass is determined by dual x-ray absorptiometry compared to baseline.

98. The method of claim 1, further comprising administering an additional therapeutically active component.

99. The method of claim 98, wherein the additional therapeutically active component is selected from the group of an additional GDF-8 inhibitor, growth factor inhibitors, immunosuppressants, anti-inflammatory agents, metabolic inhibitors, enzyme inhibitors, cytotoxic agents, and cytostatic agents.

100. The method of claim 99, wherein the additional GDF-8 inhibitor is an anti-GDF-8 antibody or antigen binding fragment thereof.

101. The method of claim 98, wherein the additional therapeutically active component is administered prior to, concurrent with, or after the administration of the GDF-8 inhibitor.

102. The method of claim 93, wherein the disease, disorder, or condition is associated with GDF-8 activity.

103. The method of claim 1, wherein the increase in lean body mass comprises an increase in total lean body mass and/or an increase in appendicular lean mass in the subject.

104. The method of claim 10, wherein the antibody or antigen-binding fragment thereof specifically binds mature GDF8 protein comprising the amino acid sequence of SEQ ID NO: 340.

105. The method of claim 10, wherein the antibody or antibody binding fragment thereof is conjugated to a therapeutic moiety.
